(19) United States
(12) Patent Application Publication
(10) Pub. No.: US 2026/0209353 A1
(43) Pub. Date: Jul. 23, 2026

(54) ANTI-CD8 ANTIBODIES AND METHODS OF USE THEREOF

(71) Applicant: Binacea Pharma, Inc., Hayward, CA (US)

(72) Inventors: Yumin DAI, Danville, CA (US); Weijun FENG, Danville, CA (US); Shouhua XIAO, Foster City, CA (US); Jie XUE, Fremont, CA (US); Halei ZHAI, Pleasant Valley, CA (US)

(73) Assignee: Binacea Pharma, Inc., Hayward, CA (US)

(21) Appl. No.: 19/143,790

(22) PCT Filed: Dec. 20, 2023
(86) PCT No.: PCT/US2023/085098

§ 371 Date: Jun. 26, 2025

Related U.S. Application Data
(60) Provisional application No. 63/477,529, filed on Dec. 28, 2022.

(51) Int. Cl. C07K 16/28; A61K 39/395; A61K 45/06

(52) U.S. Cl. CPC C07K 16/2815; A61K 39/3955; A61K 45/06; C07K 2317/24; C07K 2317/569; C07K 2317/92; C07K 2319/30

(57) ABSTRACT

The present disclosure provides anti-CD8 antibodies with a single heavy chain variable domain that specifically binds to human CD8 protein and which are capable of binding to CD8+ immune cells, with or without increasing, decreasing, inhibiting, and/or blocking immune regulatory effects mediated by CD8, including the immune response mediated by activated CD8+ T cells. The present disclosure also provides methods of using the anti-CD8 antibodies (and compositions thereof) to treat diseases and conditions mediated by activated CD8+ T cells in the immune response, including cancer, autoimmune disorders, and viral infections.

### CROSS REFERENCE TO RELATED APPLICATIONS

The present application is a national stage application under 35 USC 371 of International Application No. PCT/US2023/085098, filed Dec. 20, 2023, which claims priority to U.S. Provisional Pat. Appl. Ser. No. 63/477,529, filed Dec. 28, 2022, each of which is hereby incorporated by reference in its entirety for all purposes.

### FIELD

The present disclosure relates generally to antibodies with a single heavy chain variable domain (VH), such as VHH antibodies, which specifically bind to CD8 expressed on CD8+ T cells and methods of using such antibodies.

### REFERENCE TO SEQUENCE LISTING

The official copy of the Sequence Listing is submitted concurrently with the specification via USPTO Patent Center as an WIPO Standard ST.26 formatted XML file with file name “17195-002PV1.xml”, a creation date of Dec. 28, 2022, and a size of 100,609 bytes. This Sequence Listing filed via USPTO Patent Center is part of the specification and is incorporated in its entirety by reference herein.

### BACKGROUND

CD8 is a transmembrane glycoprotein that acts as a co-receptor with the T-cell receptor (TCR) to mediate T cell signaling that promotes cytotoxic T cell-antigen interactions. CD8 is expressed on the surface of cytotoxic T cells and binds to the major histocompatibility complex (MHC) class I protein. The extracellular domain of the CD8α isoform binds to the α3 portion of Class I MHC and this binding affinity keeps the cytotoxic T cell and the target cell bound closely during antigen-specific activation.

Cytotoxic T cells with CD8 surface protein are called CD8+ T cells. The CD8 co-receptor also facilitates T cell signaling via the cytoplasmic domain of the transmembrane CD8 receptor binding to Lck (lymphocyte-specific protein tyrosine kinase). Lck phosphorylates the cytoplasmic domain of the TCR complex which initiates a cascade of phosphorylation events resulting in activation of transcription factors including NFAT, NF-κB, and AP-1. CD8+ T cells also have the ability to make some cytokines, such as TNF-α and IFN-γ, with antitumor and antimicrobial effects.

Cytotoxic CD8+ T cells are known play an important role in the immune response against cancer. Tumors, however, have mechanisms that can defeat the CD8+ T cell immune response, such as the production of immunosuppressive cytokines, or immune checkpoint molecules, such as PD-1, CTLA4, LAG3.

There remains a need for improved antibodies that bind CD8 expressed on CD8+ T cells for uses in diagnostics, imaging, and as immunotherapeutic agents that can effectively modulate the immune response mediated by activated CD8+ T cells.

### SUMMARY

The present disclosure provides antibodies with a single heavy chain variable domain (VH), such as VHH antibodies, that specifically bind with high affinity to human CD8, including CD8 expressed on CD8+ T cells. The antibodies are capable of binding to CD8+ immune cells, with or without increasing, decreasing, inhibiting, and/or fully-blocking immune regulatory effects mediated by CD8, including the immune response mediated by activated CD8+ T cells. The antibodies are also able to function as a binding engager for other immune modulators (stimulators or inhibitors), such as cytokines such as but not limited to IL-2, TNFα, INFγ, and co-stimulators, such as but not limited to, 4-1BB, OX40, CD27 etc. to regulate immune functions of CD8+ T cells. The present disclosure also provides compositions for and methods of treating diseases and conditions mediated by activated CD8+ T cells.

In at least one embodiment, the present disclosure provides an anti-CD8 antibody comprising a single heavy chain variable domain (VH) comprising a first heavy chain complementarity region (CDR-H1), a second heavy chain complementarity region (CDR-H2), and a third heavy chain complementarity region (CDR-H3), wherein CDR-H1, CDR-H2, and CDR-H3, respectively, have amino acid sequences of: SEQ ID NO: 3, 4, and 5; SEQ ID NO: 7, 8, and 9; SEQ ID NO: 11, 12, and 13; SEQ ID NO: 15, 16, and 17; SEQ ID NO: 19, 20, and 21; SEQ ID NO: 23, 24, and 25; SEQ ID NO: 27, 28, and 29; SEQ ID NO: 31, 32, and 33; SEQ ID NO: 35, 36, and 37; SEQ ID NO: 39, 40, and 41; SEQ ID NO: 43, 44, and 45; SEQ ID NO: 47, 48, and 49; SEQ ID NO: 51, 52, and 53; SEQ ID NO: 55, 56, and 57; SEQ ID NO: 59, 60, and 61; SEQ ID NO: 63, 64, and 65; SEQ ID NO: 67, 68, and 69; SEQ ID NO: 71, 72, and 73; or SEQ ID NO: 75, 76, and 77.

In at least one embodiment of the anti-CD8 antibodies of the present disclosure, the antibody comprises a heavy chain variable domain (VH) amino acid sequence having at least 90% identity to a sequence selected from SEQ ID NO: 2, 6, 10, 14, 18, 22, 26, 30, 34, 38, 42, 46, 50, 54, 58, 62, 66, 70, and 74.

In at least one embodiment of the anti-CD8 antibodies of the present disclosure, the antibody is humanized; optionally, wherein the humanized antibody comprises a heavy chain variable domain (VH) amino acid sequence having at least 90% identity to a sequence selected from SEQ ID NO: 78, 78, 79, 80, 81, 82, 83, 84, 85, 86, 87, 88, 89, 90, 91, 92, 93, 94, 95, 96, 97, 98, 99, 100, 101, 102, 103, 104, and 105.

In at least one embodiment of the anti-CD8 antibodies of the present disclosure, the antibody is an antibody fragment; optionally, wherein the antibody fragment is selected from: a VHH antibody, a scFv antibody, a single-domain antibody, a Fv fragment, a Fab fragment, Fab′ fragment, Fab′-SH fragment, a F(ab′)₂ fragment, a diabody, a linear antibody, a single-armed antibody, and a single-chain antibody.

In at least one embodiment of the anti-CD8 antibodies of the present disclosure, the antibody comprises a fusion; optionally, wherein the fusion comprises a portion or all of an immunoglobulin Fc region, a cytokine protein, and/or another antibody. In at least one embodiment, the fusion comprises a polypeptide linker; optionally, wherein the polypeptide linker comprises an amino acid sequence selected from (GGGGS)ₙ, (SSSSG)ₙ, (GGGG)(SGGGG)ₙ, (EAAAK)ₙ, and (XP)ₙ, ENLYFQ(−G/S), where n is 1 to 10.

In at least one embodiment of the anti-CD8 antibodies of the present disclosure, the antibody is characterized by one or more of the following properties:
(a) binds to hu-CD8 with a binding affinity of 1×10⁻⁸ M or less, 1×10⁻⁹ M or less, 1×10⁻¹⁰ M or less, or 1×10⁻¹¹ M or less; optionally, wherein the binding affinity is measured by equilibrium dissociation constant (K_{D}) to a hu-CD8 polypeptide of SEQ ID NO: 1;
(b) binds to cyno-CD8 with a binding affinity of 1×10⁻⁸ M or less, 1×10⁻⁹ M or less, 1×10⁻¹⁰ M or less, or 1×10⁻¹¹ M or less; optionally, wherein the binding affinity is measured by equilibrium dissociation constant (K_{D}) to a cyno-CD8 polypeptide of SEQ ID NO: 108; and/or
(c) binds to CD8+ cells with an EC₅₀ of 2.5 nM or less, 1 nM or less, 0.5 nM or less, or 0.25 nM or less.

In at least one embodiment, the disclosure further provides an anti-CD8 antibody that specifically binds to the same epitope as the anti-CD8 antibody of clone 3A8.

In at least one embodiment the present disclosure also provides a polynucleotide or a vector encoding an anti-CD8 antibody of the present disclosure. Also provided, is a host cell comprising a polynucleotide or vector encoding an anti-CD8 antibody of the present disclosure; optionally, wherein the host cell is selected from a Chinese hamster ovary (CHO) cell, a myeloma cell (e.g., Y0, NS0, Sp2/0), a monkey kidney cell (COS-7), a human embryonic kidney line (293), a baby hamster kidney cell (BHK), a mouse Sertoli cell (e.g., TM4), an African green monkey kidney cell (VERO-76), a human cervical carcinoma cell (HELA), a canine kidney cell, a human lung cell (W138), a human liver cell (Hep G2), a mouse mammary tumor cell, a TR1 cell, a Medical Research Council 5 (MRC 5) cell, and a FS4 cell. The present disclosure further provides a method of producing an antibody comprising culturing a host cell of the present disclosure so that an anti-CD8 antibody is produced.

The present disclosure also provides a pharmaceutical composition comprising an anti-CD8 antibody of the present disclosure and a pharmaceutically acceptable carrier. In at least one embodiment, the pharmaceutical composition further comprises a chemotherapeutic agent or another antibody comprising a specificity for an immune checkpoint molecule.

The present disclosure also provides uses of the anti-CD8 antibodies. In at least one embodiment, the present disclosure provides a method of treating a CD8 mediated disease in a subject, the method comprising administering to the subject a therapeutically effective amount of an anti-CD8 antibody of the present disclosure or administering to the subject a therapeutically effective amount of a pharmaceutical composition comprising an anti-CD8 antibody of the present disclosure.

In at least one embodiment of the methods of treatment of the present disclosure, the disease is a cancer; optionally, wherein the cancer is selected from colorectal cancer, pancreatic cancer, ovarian cancer, liver cancer, renal cancer, breast cancer, lung cancer, esophageal and gastric cancer, head and neck cancer, cervical cancer, prostate cancer, melanoma, bladder cancer, oral cancer, or hematological malignancies.

In at least one embodiment of the methods of treatment of the present disclosure, the disease is an autoimmune disease; optionally, wherein the autoimmune disease is selected from Crohn's disease, ulcerative colitis, celiac disease, systemic lupus erythematosus, psoriatic arthritis, rheumatoid arthritis, Sjogren's syndrome, type 1 diabetes, atopic dermatitis, psoriasis, and multiple sclerosis.

### BRIEF DESCRIPTION OF THE FIGURES

A better understanding of the novel features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1A, FIG. 1B, FIG. 1C, FIG. 1D, FIG. 1E, FIG. 1F, FIG. 1G, FIG. 1H, FIG. 1I, FIG. 1J, and FIG. 1K depict exemplary size exclusion-high-performance liquid chromatography (SEC-HPLC) profiles of anti-CD8 VHH antibodies in Groups 1~4 as described in Example 6.

FIG. 2 depicts plots of exemplary results of flow cytometry analysis of anti-human CD8A VHH-Fc antibodies (clones 3D12, 3A8, 4H5, 4C9, and 3G7) binding to human HEK blue cells stably expressing human CD8A as measured in the whole cell binding assay described in Example 10. Bound antibodies were detected using FITC conjugated secondary antibodies before the cells were passed through a flow cytometer.

### DETAILED DESCRIPTION

The present disclosure provides antibodies with a single heavy chain variable domain (VH), such as VHH antibodies, that specifically bind to human CD8 with high affinity and thereby modulate, by itself or with another moiety, the function of CD8 as a cell surface receptor involved in immune regulation, particularly the function of CD8+ T cells in the immune response against human diseases, such as tumors, autoimmune diseases, and/or viral infection. Accordingly, it is contemplated that any of the anti-CD8 antibodies of the present disclosure can be used in pharmaceutical compositions as therapeutics for treatment of such diseases. Further, it is contemplated that the anti-CD8 antibodies of the present disclosure can be used as a therapeutic in combination, or as a fusion, with other therapeutics, such as antibodies that target immune checkpoint molecules including, but not limited to, PD1, CTLA4, LAG3, and/or cytokines, such as interleukin-2.

### Overview of Terminology and Techniques

For the descriptions herein and the appended claims, the singular forms “a”, and “an” include plural referents unless the context clearly indicates otherwise. Thus, for example, reference to “a protein” includes more than one protein, and reference to “a compound” refers to more than one compound. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

Where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding (i) either or (ii) both of those included limits are also included in the invention. For example, “1 to 50,” includes “2 to 25,” “5 to 20,” “25 to 50,” “1 to 10,” etc.

Generally, the nomenclature used herein and the techniques and procedures described herein include those that are well understood and commonly employed by those of ordinary skill in the art, such as the common techniques and methodologies described in Sambrook et al., Molecular Cloning—A Laboratory Manual (2nd Ed.), Vols. 1-3, Cold Spring Harbor Laboratory, Cold Spring Harbor, N.Y., 1989 (hereinafter “Sambrook”); Current Protocols in Molecular Biology, F. M. Ausubel et al., eds., Current Protocols, a joint venture between Greene Publishing Associates, Inc. and John Wiley & Sons, Inc. (supplemented through 2011) (hereinafter “Ausubel”); Antibody Engineering, Vols. 1 and 2, R. Kontermann and S. Dubel, eds., Springer-Verlag, Berlin and Heidelberg (2010); Monoclonal Antibodies: Methods and Protocols, V. Ossipow and N. Fischer, eds., 2nd Ed., Humana Press (2014); Therapeutic Antibodies: From Bench to Clinic, Z. An, ed., J. Wiley & Sons, Hoboken, N.J. (2009); and Phage Display, Tim Clackson and Henry B. Lowman, eds., Oxford University Press, United Kingdom (2004).

All publications, patents, patent applications, and other documents referenced in this disclosure are hereby incorporated by reference in their entireties for all purposes to the same extent as if each individual publication, patent, patent application or other document were individually indicated to be incorporated by reference herein for all purposes.

Unless defined otherwise, all technical and scientific terms used herein have the same meaning as commonly understood by one of ordinary skill in the art to which the present invention pertains. It is to be understood that the terminology used herein is for the purpose of describing particular embodiments only and is not intended to be limiting. For purposes of interpreting this disclosure, the following description of terms will apply and, where appropriate, a term used in the singular form will also include the plural form and vice versa.

“CD8” as used herein, refers to the cluster of differentiation 8 (CD8) transmembrane glycoprotein that serves as a co-receptor for the T cell receptor, and encompasses the full-length and portions of the full-length CD8 proteins of human, cynomolgus monkey (herein referred to in some cases as “cyno”), rhesus monkey, and their various isoforms. The human CD8 protein is a dimer, consisting of a pair of CD8 chains, including CD8 alpha (or “CD8A”) and CD8 beta (or “CD8B”) chains. The term “human CD8” encompasses CD8A/CD8A homodimer, CD8A/CD8B heterodimer, CD8A chain, CD8B chain, or portions thereof, such as extracellular domains. “CD8A” and “CD8a” are used interchangeably herein, and “CD8B” and “CD8b” are used interchangeably herein. An exemplary sequence of the human CD8A chain extracellular domain (ECD) is provided in Table 3 and the accompanying Sequence Listing.

“CD8 mediated condition” or “CD8 mediated disease,” as used herein, encompasses any medical condition associated with or effected by CD8, or a therapeutic effect mediated by or afforded by CD8+ cells (including, but not limited to CD8+ T cells, NK cells, NKT cells). For example, specific binding to CD8 expressed on cell surfaces can alter activation of CD8+ lymphocytes (e.g., T cells). Accordingly, CD8 mediated diseases can include, but are not limited to, any disease or condition mediated by and/or responsive to agonists (or activators), or antagonists (or inhibitors) of CD8 expressing cells, including but not limited to autoimmune disorders and cancers. Specific exemplary autoimmune disorders and cancers are provided elsewhere herein.

“Antibody,” as used herein, refers to a molecule comprising one or more polypeptide chains that specifically binds to, or is immunologically reactive with, a particular antigen. Exemplary antibodies include monoclonal antibodies, polyclonal antibodies, chimeric antibodies, humanized antibodies, human antibodies, multispecific antibodies (e.g., bispecific antibodies), monovalent antibodies (e.g., single-arm antibodies), multivalent antibodies, single-chain antibodies, heavy chain (only) antibodies (e.g., sdAb, or VHH, IgNAR, nanobody (or nanoAb)), antigen-binding fragments (e.g., Fab′, F(ab′)₂, Fab, Fv, rIgG, and scFv fragments), antibody fusions, and synthetic antibodies (or antibody mimetics).

“Anti-CD8 antibody” or “antibody that binds CD8” refers to an antibody that binds CD8 with sufficient affinity such that the antibody is useful as a diagnostic and/or therapeutic agent in targeting CD8. In some embodiments, the extent of binding of an anti-CD8 specific antibody to an unrelated, non-CD8 antigen is less than about 20%, less than about 15%, less than about 10%, or less than about 5% of the binding of the antibody to CD8 as measured, e.g., by a radioimmunoassay (RIA) or surface plasmon resonance (SPR). In some embodiments, an antibody that binds to CD8 has a dissociation constant (K_{D}) of <1 μM, <100 nM, <10 nM, <1 nM, <0.1 nM, <0.01 nM, or <1 μM (e.g., 10⁻⁸ M or less, e.g., from 10⁻⁸ M to 10⁻¹³ M, e.g., from 10⁻⁹ M to 10⁻¹³ M).

“Full-length antibody,” “intact antibody,” or “whole antibody” are used herein interchangeably to refer to an antibody having a structure substantially similar to a native antibody structure or having heavy chains that contain an Fc region as defined herein.

“Antibody fragment” refers to a portion of a full-length antibody which is capable of binding the same antigen as the full-length antibody. Examples of antibody fragments include, but are not limited to, VHH, single-domain antibodies, Fv, Fab, Fab′, Fab′-SH, F(ab′)₂ fragments, diabodies; linear antibodies; monovalent, or single-armed antibodies; single-chain antibody molecules (e.g., scFv); and multispecific antibodies formed from antibody fragments.

“Heavy chain antibody,” “heavy chain-only antibody,” or “HCAb,” as used herein, refers to a functional antibody, which comprises two heavy chains, but lacks two light chains usually found in 4-chain antibodies. Camelid animals (such as camels, llamas, or alpacas) are known to produce HCAbs.

“Single-domain antibody” or “sdAb,” as used herein, refers to a single antigen-binding domain having three complementarity determining regions (CDRs). The sdAb alone is capable of binding to the antigen without pairing with a corresponding CDR-containing polypeptide. A camelid sdAb is one of the smallest known antigen-binding antibody fragments (see, e.g., Hamers-Casterman et al., Nature 363:446-8 (1993); Greenberg et al., Nature 374:168-73 (1995); Hassanzadeh-Ghassabeh et al., Nanomedicine (Lond), 8:1013-26 (2013)). “VHHs” (defined below) are a type of single-domain antibody that are engineered from camelid HCAbs.

“VHH,” or “variable domain of the heavy chain of a heavy chain antibody” refers to a single chain including a heavy chain variable domain of an antibody. A VHH typically has the following structure from the N-terminus to the C-terminus: FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3. VHH molecules can be derived from antibodies raised in Camelidae species, for example, camel, llama, vicuna, dromedary, alpaca, and guanaco, or generated from synthetic libraries.

“VHH antibody” or “heavy chain antibody” or “heavy chain only antibody” refers to a single chain including a heavy chain variable domain of an antibody and an Fc region, with a hinge or other linker of amino acids with natural or synthetic sequence in between. The “single chain” could form dimer, such as when fused with Fc region that is a dimer.

“Class” of an antibody refers to the type of constant domain or constant region possessed by its heavy chain. There are five major classes of antibodies: IgA, IgD, IgE, IgG, and IgM, and several of these are further divided into subclasses (isotypes), e.g., IgG1, IgG2, IgG3, IgG4, IgAQ1, and IgA2. The heavy chain constant domains that correspond to the different classes of immunoglobulins are called α, δ, ε, γ, and μ, respectively.

“Variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three hypervariable regions (HVRs) (see, e.g., Kindt et al., Kuby Immunology, 6th ed., W.H. Freeman and Co., page 91). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively (see, e.g., Portolano et al., J. Immunol. 150:880-887 (1993); Clarkson et al., Nature 352:624-628 (1991)).

“Hypervariable region” or “HVR,” as used herein, refers to each of the regions of an antibody variable domain which are hypervariable in sequence and/or form structurally defined loops (“hypervariable loops”). Generally, native antibodies comprise four chains with six HVRs; three in the heavy chain variable domain, V_{H} (HVR-H1, HVR-H2, HVR-H3), and three in the light chain variable domain, V_{L} (HVR-L1, HVR-L2, HVR-L3). The HVRs generally comprise amino acid residues from the hypervariable loops and/or from the “complementarity determining regions” (CDRs). A number of hypervariable region delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM hypervariable regions represent a compromise between the Kabat CDRs and Chothia structural loops, and are used by Oxford Molecular's AbM antibody modeling software. The “contact” hypervariable regions are based on an analysis of the available complex crystal structures. The residues from each of these hypervariable regions are noted in the Table 1 below.

TABLE 1

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B¹ | H26-H35B¹ | H26-H32¹ | H30-H35B¹ |
| | H31-H35² | H26-H35² | H26-H32² | H30-H35² |
| H2 | H50-H65 | H50-H58 | H53-H55 | H47-H58 |
| H3 | H95-H102 | H95-H102 | H96-H101 | H93-H101 |

¹Kabat numbering
²Chothia numbering

Unless otherwise indicated, HVR residues and other residues in the variable domain (e.g., FR residues) are numbered herein according to Kabat et al., supra.

Hypervariable regions, as used herein, may include extended or alternative hypervariable regions as follows: 26-35 or 30-35 (H1), 50-61, 50-65 or 49-65 (H2) and 93-102, 94-102, or 95-102 (H3) in the V_{H} domain; and 24-36 or 24-34 (L1), 46-56 or 50-56 (L2) and 89-97 or 89-96 (L3) in the V_{L} domain. The variable domain residues are numbered according to Kabat et al., supra, for each of these definitions.

“Complementarity determining region,” or “CDR,” as used herein, refers to the regions within the HVRs of the variable domain which have the highest sequence variability and/or are involved in antigen recognition. Generally, native antibodies comprise four chains with six CDRs; three in the heavy chain variable domains, V_{H} (H1, H2, H3), and three in the light chain variable domains, V_{L} (L1, L2, L3). Generally, a VHH antibody comprises just the CDRs of the heavy chain (VH (H1, H2, H3). Exemplary CDRs (numbered according to Kabat et al., supra) occur at the following amino acid residue positions of the VH and VL domains: CDR-H1 at 31-35; CDR-H2 at 50-61; CDR-H3 at 95-102; CDR-L1 at 24-34; CDR-L2 at 50-56; and CDR-L3 at 89-97.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in V_{H} (or V_{L}): FR1-H1(L1)-FR2-H2 (L2)-FR3-H3 (L3)-FR4.

“Native antibody” refers to a naturally occurring immunoglobulin molecule. For example, native IgG antibodies are heterotetrameric glycoproteins of about 150,000 Daltons, composed of two identical light chains and two identical heavy chains that are disulfide-bonded. From N- to C-terminus, each heavy chain has a variable region (V_{H}), also called a variable heavy domain or a heavy chain variable domain, followed by three constant domains (CH1, CH2, and CH3). Similarly, from N- to C-terminus, each light chain has a variable region (V_{L}), also called a variable light domain or a light chain variable domain, followed by a constant light (CL) domain. The light chain of an antibody may be assigned to one of two types, called kappa (κ) and lambda (λ), based on the amino acid sequence of its constant domain.

“Monoclonal antibody” as used herein refers to an antibody obtained from a substantially homogeneous population of antibodies, i.e., the individual antibodies comprising the population are identical and/or bind the same epitope, except for possible variant antibodies (e.g., variant antibodies contain mutations that occur naturally or arise during production of a monoclonal antibody, and generally are present in minor amounts). In contrast to polyclonal antibody preparations, which typically include different antibodies directed against different determinants (epitopes), each monoclonal antibody of a monoclonal antibody preparation is directed against a single determinant on an antigen. Thus, the term “monoclonal” indicates the character of the antibody as being obtained from a substantially homogeneous population of antibodies and is not to be construed as requiring production of the antibody by any particular method. For example, the monoclonal antibodies to be used may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“Chimeric antibody” refers to an antibody in which a portion of the heavy and/or light chain is derived from a particular source or species, while the remainder of the heavy and/or light chain is derived from a different source or species.

“Humanized antibody” refers to a chimeric antibody comprising amino acid sequences from non-human HVRs and amino acid sequences from human FRs. In certain embodiments, a humanized antibody will comprise substantially all of at least one, and typically two, variable domains, in which all or substantially all of the HVRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

“Human antibody” refers to an antibody which possesses an amino acid sequence corresponding to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

“Human consensus framework” is a framework which represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In some embodiments, for the V_{L}, the subgroup is subgroup kappa I as in Kabat et al., supra. In some embodiments, for the V_{H}, the subgroup is subgroup III as in Kabat et al., supra.

“Acceptor human framework” as used herein is a framework comprising the amino acid sequence of a light chain variable domain (V_{L}) framework or a heavy chain variable domain (V_{H}) framework derived from a human immunoglobulin framework or a human consensus framework. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the V_{L} acceptor human framework is identical in sequence to the V_{L} human immunoglobulin framework sequence or human consensus framework sequence.

“Fc region,” or “fragment crystallizable region” refers to a dimer complex comprising the C-terminal polypeptide sequences of an immunoglobulin heavy chain, wherein a C-terminal polypeptide sequence is that which is obtainable by papain digestion of an intact antibody. The Fc region of an immunoglobulin generally includes the heavy chain CH2 and CH3 domains, and optionally the CH4 domain. The Fc region may comprise native or variant Fc sequences. Although the boundaries of the Fc sequence of an immunoglobulin heavy chain may vary, the human IgG heavy chain Fc sequence is usually defined to stretch from an amino acid residue at about position Cys226, or from about position Pro230, to the carboxyl-terminus of the Fc sequence.

“Fc receptor” or “FcR,” refers to a receptor that binds to the Fc region of an antibody. In some embodiments, an FcR is a native human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain, (see, e.g., Daeron, Annu. Rev. Immunol. 15:203-234 (1997)). FcR, as used herein, also includes the neonatal receptor, FcRn, which is responsible for the transfer of maternal IgGs to the fetus (Guyer et al, J. Immunol. 117:587 (1976) and Kim et al, J. Immunol. 24:249 (1994)) and regulation of homeostasis of immunoglobulins. FcRs are reviewed, for example, in Ravetch and Kinet, Annu. Rev. Immunol 9:457-92 (1991); Capel et al, Immunomethods 4:25-34 (1994); and de Haas et al, J. Lab. Clin. Med. 126:330-41 (1995).

“Multivalent antibody,” as used herein, is an antibody comprising three or more antigen binding sites. The multivalent antibody is preferably engineered to have the three or more antigen binding sites and is generally not a native sequence IgM or IgA antibody.

“Multispecific antibody” is an antibody having at least two different binding sites, each site with a different binding specificity. A multispecific antibody can be a full length antibody or an antibody fragment, and the different binding sites may bind each to a different antigen or the different binding sites may bind to two different epitopes of the same antigen.

“Fv fragment” refers to an antibody fragment which contains a complete antigen recognition and binding site. This region consists of a dimer of one heavy and one light chain variable domain in tight association, which can be covalent in nature, for example in scFv. It is in this configuration that the three HVRs of each variable domain interact to define an antigen binding site on the surface of the V_{H}-V_{L} dimer. Collectively, the six HVRs or a subset thereof confer antigen binding specificity to the antibody. However, even a single variable domain (or half of an Fv comprising only three HVRs specific for an antigen) has the ability to recognize and bind antigen, although usually at a lower affinity than the entire binding site.

“Fab fragment’ refers to an antibody fragment that contains a variable and constant domain of the light chain and a variable domain and the first constant domain (CH1) of the heavy chain. “F(ab′)₂ fragments” comprise a pair of Fab fragments which are generally covalently linked near their carboxy termini by hinge cysteines between them. Other chemical couplings of antibody fragments also are known in the art.

“Antigen binding arm,” as used herein, refers to a component of an antibody that has an ability to specifically bind a target molecule of interest. Typically the antigen binding arm is a complex of immunoglobulin polypeptide sequences, e.g., HVR and/or variable domain sequences of an immunoglobulin light and heavy chain.

“Single-chain Fv” or “scFv” refer to antibody fragments comprising the V_{H} and V_{L} domains of an antibody, wherein these domains are present in a single polypeptide chain. Generally, an Fv polypeptide further comprises a polypeptide linker between the V_{H} and V_{L} domains which enables the scFv to form the desired antigen binding structure.

“Diabodies” refers to small antibody fragments with two antigen-binding sites, which fragments comprise a heavy chain variable domain (V_{H}) connected to a light chain variable domain (V_{L}) in the same polypeptide chain (V_{H} and V_{L}). By using a linker that is too short to allow pairing between the two domains on the same chain, the domains are forced to pair with the complementary domains of another chain and create two antigen-binding sites.

“Linear antibodies” refers to the antibodies described in Zapata et al., Protein Eng., 8 (10): 1057-1062 (1995). Briefly, these antibodies comprise a pair of tandem Fd segments (VH-CH1-VH-CH1) which, together with complementary light chain polypeptides, form a pair of antigen binding regions. Linear antibodies can be bispecific or monospecific.

“Naked antibody” refers to an antibody that is not conjugated to a heterologous moiety (e.g., a cytotoxic moiety) or radiolabel.

“Affinity” refers to the strength of the sum total of noncovalent interactions between a single binding site of a molecule (e.g., an antibody) and its binding partner (e.g., an antigen). “Binding affinity” refers to intrinsic binding affinity which reflects a 1:1 interaction between members of a binding pair (e.g., antibody and antigen). The affinity of a molecule X for its partner Y can generally be represented by the equilibrium dissociation constant (K_{D}). Affinity can be measured by common methods known in the art, including those described herein. Specific illustrative and exemplary embodiments for measuring binding affinity are described in the following.

“Binds specifically” or “specific binding” refers to binding of an antibody to an antigen with an affinity value of no more than about 1×10⁻⁷ M. In some embodiments, an antibody may have a secondary affinity for an antigen other than the antigen to which it binds specifically, where “secondary affinity” will generally refer to binding of an antibody to a secondary antigen with an affinity value of more than about 10 nM as described elsewhere herein. Where an antibody may have a secondary affinity for a secondary antigen, such an antibody will nevertheless bind specifically to the primary antigen.

“Affinity matured” antibody refers to an antibody with one or more alterations in one or more HVRs, compared to a parent antibody which does not possess such alterations, such alterations resulting in an improvement in the affinity of the antibody for antigen.

“Functional antigen binding site” of an antibody is one which is capable of binding a target antigen. The antigen binding affinity of the antigen binding site is not necessarily as strong as the parent antibody from which the antigen binding site is derived, but the ability to bind antigen must be measurable using any one of a variety of methods known for evaluating antibody binding to an antigen.

“Isolated antibody” refers to an antibody which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic methods (e.g., ion exchange or reverse phase HPLC). For review of methods for assessment of antibody purity, see, e.g., Flatman et al., J. Chromatogr. B 848:79-87.

“Substantially similar” or “substantially the same,” as used herein, refers to a sufficiently high degree of similarity between two numeric values (for example, one associated with a test antibody and the other associated with a reference antibody), such that one of skill in the art would consider the difference between the two values to be of little or no biological and/or statistical significance within the context of the biological characteristic measured by said values (e.g., K_{D} values).

“Substantially different,” as used herein, refers to a sufficiently high degree of difference between two numeric values (generally one associated with a molecule and the other associated with a reference molecule) such that one of skill in the art would consider the difference between the two values to be of statistical significance within the context of the biological characteristic measured by said values (e.g., K_{D} values).

“Effector functions” refer to those biological activities attributable to the Fc region of an antibody, which vary with the antibody isotype. Examples of antibody effector functions include: Clq binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

“Immunoconjugate” refers to an antibody conjugated to one or more heterologous molecule(s), including but not limited to a cytotoxic agent.

“Fusion,” or “fusion protein,” as used herein, refers to two or more protein and/or polypeptide molecules that are linked (or “fused”) in a configuration that does not occur naturally. Fusion proteins of the present disclosure include “antibody fusions” that comprise an anti-CD8 VHH antibody covalently linked through a polypeptide linker sequence to a cytokine protein and/or another antibody.

“Polypeptide linker” or “linker sequence” as used herein refers to a chain of two or more amino acids with each end of the chain covalently attached to a different polypeptide molecule, thereby functioning to conjugate or fuse the different polypeptides. Typically, polypeptide linkers comprise polypeptide chains of 5 to 30 amino acids. A wide range of polypeptide linkers are known in the art and can be used in the compositions and methods of the present disclosure. Exemplary polypeptide linkers include in the compositions and methods of the present disclosure include, but are not limited to (GGGGS)ₙ, (SSSSG)ₙ, (GGGG)(SGGGG)ₙ, (EAAAK)ₙ, (XP)ₙ, ENLYFQ(−G/S), typically, where n is 1 to 10. A wide range of polypeptide linkers are known in the art and can be used in fusions of the anti-CD8 VHH antibodies of the present disclosure.

“Treatment,” “treat” or “treating” refers to clinical intervention in an attempt to alter the natural course of a disorder in the individual being treated and can be performed either for prophylaxis or during the course of clinical pathology. Desired results of treatment can include, but are not limited to, preventing occurrence or recurrence of the disorder, alleviation of symptoms, diminishment of any direct or indirect pathological consequences of the disorder, preventing metastasis, decreasing the rate of progression, amelioration or palliation of a disease state, and remission or improved prognosis. For example, treatment can include administration of a therapeutically effective amount of pharmaceutical formulation comprising an anti-CD8 antibody to a subject to delay development or slow progression of a disease or condition mediated by CD8 or disease or condition in which CD8 may play a role in the pathogenesis and/or progression.

“Pharmaceutical formulation” refers to a preparation in a form that allows the biological activity of the active ingredient(s) to be effective, and which contain no additional components which are toxic to the subjects to which the formulation is administered. A pharmaceutical formulation may include one or more active agents. For example, a pharmaceutical formulation may include an anti-CD8 antibody as the sole active agent of the formulation or may include an anti-CD8 antibody and one or more additional active agents, such as e.g., an immune checkpoint inhibitor.

By “sole active agent”, as used herein, is meant that the agent referred to is the only agent present in the formulation, or used in the therapy, that provides, or would be expected to provide, the relevant pharmacological effect to treat the subject for the condition, consistent with the description of “treatment” as provided herein. A pharmaceutical formulation comprising a sole active agent does not exclude the presence of one or more non-active agents, such as e.g., a pharmaceutically acceptable carrier, in the formulation. A “non-active agent” is an agent that would not be expected to provide, or otherwise significantly contribute to, the relevant pharmacological effect intended to treat the subject for the condition.

“Pharmaceutically acceptable carrier” refers to an ingredient in a pharmaceutical formulation, other than an active ingredient, which is nontoxic to the subject to whom it is administered. A pharmaceutically acceptable carrier includes, but is not limited to, a buffer, excipient, stabilizer, or preservative.

“Therapeutically effective amount” refers to the amount of an active ingredient or agent (e.g., a pharmaceutical formulation) to achieve a desired therapeutic or prophylactic result, e.g., to treat or prevent a disease, disorder, or condition in a subject. In the case of a CD8 mediated disease or condition, the therapeutically effective amount of the therapeutic agent is an amount that reduces, prevents, inhibits, and/or relieves to some extent one or more of the symptoms associated with the disease, disorder, or condition. For cancer therapy, efficacy in vivo can, for example, be measured by assessing the growth of a primary tumor, occurrence and/or growth of secondary tumor(s), occurrence and/or number of metastases, duration, severity, and/or recurrence of symptoms, the response rate (RR), duration of response, and/or quality of life.

“Concurrently,” as used herein, refers to administration of two or more therapeutic agents, where at least part of the administration overlaps in time. Accordingly, concurrent administration includes a dosing regimen when the administration of one or more agent(s) continues after discontinuing the administration of one or more other agent(s).

“Individual” or “subject” refers to a mammal, including but not limited to, domesticated animals (e.g., cows, sheep, cats, dogs, and horses), primates (e.g., humans and non-human primates such as monkeys), rabbits, and rodents (e.g., mice and rats).

### Detailed Description of Various Embodiments

### I. CD8 Polypeptides

Human CD8 (“hu-CD8”) is a transmembrane glycoprotein that is expressed on the surface of cells, notably, myeloid cells and T cells. The hu-CD8 Uniprot sequence P01732 encodes a 235 amino acid polypeptide. The 161 amino acid sequence corresponding to the extra-cellular domain (ECD) segment of the hu-CD8 (positions 22-182) is set forth herein as SEQ ID NO: 1, in Table 2 below. A recombinantly prepared ECD segment of cynomolgus monkey CD8 (“cyno-CD8”) is set forth as SEQ ID NO: 108 in Table 2 below. Table 2 below provides a summary description of the sequences of the various CD8 polypeptides of the present disclosure, and their sequence identifiers. The sequences also are included in the accompanying Sequence Listing.

TABLE 2
CD8 sequences

| | | SEQ |
|---|---|---|
| | | ID |
| Name | Sequence | NO: |
| Human CD8A ECD | SQFRVSPLDRTWNLGETVELKCQ | 1 |
| (amino acids | VLLSNPTSGCSWLFQPRGAAASP | |
| 22-182 of | TFLLYLSQNKPKAAEGLDTQRFS | |
| Uniprot P01732) | GKRLGDTFVLTLSDERRENEGYY | |
| | FCSALSNSIMYFSHFVPVFLPAK | |
| | PTTTPAPRPPTPAPTIASQPLSL | |
| | RPEACRPAAGGAVHTRGLDFACD | |
| Cyno CD8A ECD | NQFRVSPLGRTWNLGETVELKCQ | 108 |
| (amino acids | VLLSNPTSGCSWLFQPRGTAARP | |
| 22-182 of | TFLLYLSQNKPKAAEGLDTORFS | |
| Uniprot | GKRLGDTFVLTLRDFRQENEGYY | |
| A0A2K5ULI8) | FCSALSNSIMYFSHFVPVFPCSE | |
| | AHHYASAATTHTGAHHRVAAPVP | |
| | APRGVPASGGGSVNTRGLDFACD | |

### II. Anti-CD8 Antibodies

In some embodiments, the present disclosure provides structures of anti-CD8 antibodies with a single heavy chain variable domain (VH) in terms of the amino acid and encoding nucleotide sequences of the various well-known immunoglobulin features (e.g., CDRs, VH domain). Table 3 below provides a summary description of anti-CD8 antibody sequences of the present disclosure, and their sequence identifiers. The sequences are included in the accompanying Sequence Listing.

TABLE 3
Anti-CD8 VHH antibody sequences

| | | SEQ |
|---|---|---|
| | | ID |
| Name | Amino acid sequence | NO: |
| Clone 3A8 VHH | QVQLQESGGGLVQAGGSLRLSCAASGFTFDEYAIGWFRQAPGKER | 2 |
| | EGVSCLRVSDGRTYYPDSVKGRFAISSDNAKNTVYLQMNNLKPED | |
| | TAVYYCASGSYYGCTVDDYDFWGQGTQVTVSS | |
| Clone 3A8 CDR1 | EYAIG | 3 |
| Clone 3A8 CDR2 | CLRVSDGRTYYPDSVKG | 4 |
| Clone 3A8 CDR3 | GSYYGCTVDDYDF | 5 |
| Clone 3D10 VHH | QVQLQESGGGLVQAGGSLRLSCAASGFTFDEYAIGWFRQAPGKER | 6 |
| | EGVSCLRVSDGRTYYPDSVKGRFAISSDNAKNTVYLQMNNLKPED | |
| | TAVYYCASGSYYGCTVHDYDFWGQGTLVTVSS | |
| Clone 3D10 CDR1 | EYAIG | 7 |
| Clone 3D10 CDR2 | CLRVSDGRTYYPDSVKG | 8 |
| Clone 3D10 CDR3 | GSYYGCTVHDYDF | 9 |
| Clone 3A4 VHH | QVQLQESGGGLVQAGGSLRLSCAASGFTFDEYAIGWFRQAPGKER | 10 |
| | EGVSCLRVSDGRTYYPDSVKGRFAISSDNAKNTVYLQMNNLKPED | |
| | TAVYYCASGSYYGCTVHDYDFWGQGTQVTVSS | |
| Clone 3A4 CDR1 | EYAIG | 11 |
| Clone 3A4 CDR2 | CLRVSDGRTYYPDSVKG | 12 |
| Clone 3A4 CDR3 | GSYYGCTVHDYDF | 13 |
| Clone 4D5 VHH | QVQLQESGGGLVQAGGSLRLSCAASGFTFNEYAIGWFRQAPGKER | 14 |
| | EGVSCLRVSDGRTYYPYSVKGRFAISNDNANNTVYLQMNNLKPED | |
| | TAVYYCASGSYYGCTVDDYDFWGQGTQVTVSS | |
| Clone 4D5 CDR1 | EYAIG | 15 |
| Clone 4D5 CDR2 | CLRVSDGRTYYPYSVKG | 16 |
| Clone 4D5 CDR3 | GSYYGCTVDDYDF | 17 |
| Clone 1B1 VHH | QVQLQESGGGLVQAGGSLKLSCAASGFTFNDYAIGWFPQAPGKER | 18 |
| | EGVSCIINIDGSTYYADSVKGRFPISSDNAKNTVYLQMNSLKPED | |
| | TAVYYCAAEGLHSCRDLYYGMDYWGKGTLVTVSS | |
| Clone 1B1 CDR1 | AIGWE | 19 |
| Clone 1B1 CDR2 | CIINIDGSTYYADSVKG | 20 |
| Clone 1B1 CDR3 | EGLHSCRDLYYGMDY | 21 |
| Clone 2G3 VHH | QVQLQESGGGLVQPGGSLKLSCAASGFTFYDYAIGWFRQAPGKER | 22 |
| | EGVSCISNIDGSTYYADSVKGRFTISTDNAKNTVYLQMNSLKPED | |
| | TAVYYCAAEGLHSCRDLYYGMDYWGKGTLVTVSS | |
| Clone 2G3 CDR1 | AIGWF | 23 |
| Clone 2G3 CDR2 | CISNIDGSTYYADSVKG | 24 |
| Clone 2G3 CDR3 | EGLHSCRDLYYGMDY | 25 |
| Clone 2G1 VHH | QVQLQESGGGLVQAGGSLRLSCAASGFTEDDYAIGWFRQAPGKER | 26 |
| | EGVSCISSIDGSTYYADSVKGRFTISRDNDKNTVYLQMNSLKPED | |
| | TAVYYCTAEGLHSCRDLYYGMDYWGKGTQVTVSS | |
| Clone 2G1 CDR1 | AIGWF | 27 |
| Clone 2G1 CDR2 | CISSIDGSTYYADSVKG | 28 |
| Clone 2G1 CDR3 | EGLHSCRDLYYGMDY | 29 |
| Clone 2E7 VHH | QVQLQESGGGLVQAGGSLRLSCAASGVPFDDYAIGWFRQAPGKER | 30 |
| | EGVSCISNIDGSTYYADSVKGRFTISSDNAKNTVYLQMNSLKPED | |
| | TAVYYCAAEGLHSCRDLYYGMDYWGKGTLVTVSS | |
| Clone 2E7 CDR1 | AIGWF | 31 |
| Clone 2E7 CDR2 | CISNIDGSTYYADSVKG | 32 |
| Clone 2E7 CDR3 | EGLHSCRDLYYGMDY | 33 |
| Clone 1B3 VHH | QVQLQESGGGLVQAGGSLRLSCAASGFTFDDYAIGWFRQAPGKER | 34 |
| | EGVSCISNIDGHTYYADSVKGRFTISSDNAKNTVYLQMNSLKPED | |
| | TAVYYCAAEGLHSCKDLYYGMDYWGKGTLVTVSS | |
| Clone 1B3 CDR1 | AIGWF | 35 |
| Clone 1B3 CDR2 | CISNIDGHTYYADSVKG | 36 |
| Clone 1B3 CDR3 | EGLHSCKDLYYGMDY | 37 |
| Clone 1E2 VHH | QVQLQESGGGLVQAGGSLRLSCAASGFTEDDYAIGWFRQAPGKER | 38 |
| | EGVSCISNIDGRTYYADSVKGRFTISSDNAKNTVYLQMNSLKPED | |
| | TAVYYCAAEGLHPCRDLYYGMDYWGKGTLVTVSS | |
| Clone 1E2 CDR1 | AIGWF | 39 |
| Clone 1E2 CDR2 | CISNIDGRTYYADSVKG | 40 |
| Clone 1E2 CDR3 | EGLHPCRDLYYGMDY | 41 |
| Clone 1D7 VHH | QVQLQESGGGLVQAGGSLRLSCAASGFTFDDYAIGWFRQAPGKER | 42 |
| | EGVSCISNIDGRTYYADSVKGRFTISSDNAKNTVYLQMNSLKPED | |
| | TAVYYCAAEGLHSCRDLYYGMDYWGKGTLVTVSS | |
| Clone 1D7 CDR1 | AIGWE | 43 |
| Clone 1D7 CDR2 | CISNIDGRTYYADSVKG | 44 |
| Clone 1D7 CDR3 | EGLHSCRDLYYGMDY | 45 |
| Clone 2G7 VHH | QVKLEESGGGLVQAGGSLRLSCAASGFTFDDYAIGWFRQAPGKER | 46 |
| | EGVSCISNIDGSTYYADSVKGRFTISSDNAKNTVYLQMNSLKPED | |
| | TAVYYCAAEGLHSCRDLYYGMDHWGKGTLVTVSS | |
| Clone 2G7 CDR1 | AIGWF | 47 |
| Clone 2G7 CDR2 | CISNIDGSTYYADSVKG | 48 |
| Clone 2G7 CDR3 | EGLHSCRDLYYGMDH | 49 |
| Clone 1A5 VHH | QVQLVESGGGLVQAGGSLRLSCAASGFTFDDYAIGWFRQAPGKER | 50 |
| | EGVSCISNIDGSTYYADSVKGRFTISSDNAKNTVYLQMNSLKPED | |
| | TAVYYCAAEGLHSCRDLYYGMDYWGKGTLVTVSS | |
| Clone 1A5 CDR1 | AIGWF | 51 |
| Clone 1A5 CDR2 | CISNIDGSTYYADSVKG | 52 |
| Clone 1A5 CDR3 | EGLHSCRDLYYGMDY | 53 |
| Clone 2B10 VHH | QVQLVESGGGLVQAGGSLRLSCAASGFTFDDYAIGWFRQAPGKER | 54 |
| | EGVSCISNIDGSTYYADSVKGRFTISSDNAKNTVYLQMNSLKPED | |
| | TAVYYCAAEGLRSCRDLYYGMDYWGKGTLVTVSS | |
| Clone 2B10 CDR1 | AIGWF | 55 |
| Clone 2B10 CDR2 | CISNIDGSTYYADSVKG | 56 |
| Clone 2B10 CDR3 | EGLRSCRDLYYGMDY | 57 |
| Clone 3C3 VHH | QVQLVDSGGGLVQAGGSLRVSCAASGLTVSSEHMGWFRQAPGKQR | 58 |
| | EFVAAISGNGGSTYYADSVKGRFTISRDNAKNTVYLQMNSLKPED | |
| | TAVYTCAADRQVWRVTSYHDYDYDSWGQGTLVTVSS | |
| Clone 3C3 CDR1 | HMGWF | 59 |
| Clone 3C3 CDR2 | AISGNGGSTYYADSVKG | 60 |
| Clone 3C3 CDR3 | DRQVWRVTSYHDYDYDS | 61 |
| Clone 4H5 VHH | QVQLQESGGGLVQAGGSLRLSCAASGLTVNSDHMAWFRQAPGKWR | 62 |
| | DFVAAISGNGGSTYYADSVKGRFTISRDDAKNTVYLQMNSLKPED | |
| | TAVYTCAADRQVWRAASYHDYDYDSWGQGTLVTVSS | |
| Clone 4H5 CDR1 | HMAWF | 63 |
| Clone 4H5 CDR2 | AISGNGGSTYYADSVKG | 64 |
| Clone 4H5 CDR3 | DRQVWRAASYHDYDYDS | 65 |
| Clone 4C9 VHH | QVKLEESGGGLVQAGDSLRLSCAASGLTVSSDHMAWFRQAPGKWR | 66 |
| | EFVAAISGNGGSTYYADSVKGRFTISRDNAKNTVYLQMNNLKPED | |
| | TAVYSCAADRQVWRVASYHDYDYDSWGQGTQVTVSS | |
| Clone 4C9 CDR1 | HMAWF | 67 |
| Clone 4C9 CDR2 | AISGNGGSTYYADSVKG | 68 |
| Clone 4C9 CDR3 | DRQVWRVASYHDYDYDS | 69 |
| Clone 3G7 VHH | QVQLQESGGGLVQAGGSLRLSCAASGSIAEIASLRAMDWYRQAPG | 70 |
| | KRRELVAHIARNDVTNYADDVKGRFTISRDNAKNAVYLQMNNLKP | |
| | EDTAVYYCNTVPWYGTWGQGTLVTVSS | |
| Clone 3G7 CDR1 | AMDWY | 71 |
| Clone 3G7 CDR2 | HIARNDVTNYADDVKG | 72 |
| Clone 3G7 CDR3 | VPWYGT | 73 |
| Clone 3D12 VHH | QVQLVESGGGLVQAGGSLRLSCAASGNIDEIASIRAMDWYRQAPG | 74 |
| | RQRELVAHIARNGITDYKDFVKGRFTISRDNAKNTVYLQMNSLKP | |
| | EDTAVYYCNTVPWYGTWGQGTQVTVSS | |
| Clone 3D12 CDR1 | AMDWY | 75 |
| Clone 3D12 CDR2 | HIARNGITDYKDFVKG | 76 |
| Clone 3D12 CDR3 | VPWYGT | 77 |
| Clone 3A8-hu01 VHH | EVQLVESGGGLVQPGGSLRLSCAASGFTFDEYAIGWFRQAPGKER | 78 |
| | EGVSCLRVSDGRTYYPDSVKGRFTISRDNSKNTVYLQMNSLRAED | |
| | TAVYYCASGSYYGCTVDDYDFWGQGTQVTVSS | |
| Clone 3A8-hu02 VHH | EVQLQESGGGLVQAGGSLRLSCAASGFTFDEYAIGWVRQAPGKER | 79 |
| | EGVSCLRVSDGRTYYPDSVKGRFTISRDNSKNTVYLQMNNLKPED | |
| | TAVYYCASGSYYGCTVDDYDFWGQGTQVTVSS | |
| Clone 3A8-hu03 VHH | EVQLQESGGGLVQPGGSLRLSCAASGFTFDEYAIGWVRQAPGKER | 80 |
| | EGVSCLRVSDGRTYYPDSVKGRFTISRDNSKNTVYLQMNSLRAED | |
| | TAVYYCASGSYYGCTVDDYDFWGQGTQVTVSS | |
| Clone 3A8-hu04 VHH | EVQLQESGGGLVQPGGSLRLSCAASGFTFDEYAIGWVRQAPGKER | 81 |
| | EGVSCLRVSDGRTYYPDSVKGRFTISTDNSKNTVYLQMNSLRAED | |
| | TAVYYCASGSYYGCTVDDYDFWGQGTQVTVSS | |
| Clone 3A8-hu05 VHH | QVQLQESGGGLVQAGGSLRLSCAASGFTFDEYAIGWVRQAPGKGL | 82 |
| | EGVSCLRVSDGRTYYPDSVKGRFAISSDNAKNTVYLQMNNLKPED | |
| | TAVYYCASGSYYGCTVDDYDFWGQGTQVTVSS | |
| Clone 3A8-hu06 VHH | EVQLQESGGGLVQPGGSLRLSCAASGFTFDEYAIGWVRQAPGKGL | 83 |
| | EGVSCLRVSDGRTYYPDSVKGRFTISRDNAKNTVYLQMNNLKPED | |
| | TAVYYCASGSYYGCTVDDYDFWGQGTTVTVSS | |
| Clone 3A8-hu07 VHH | EVQLQESGGGLVQAGGSLRLSCAASGFTFDEYAIGWVRQAPGKGL | 84 |
| | EGVSCLRVSDGRTYYPDSVKGRFTISRDNSKNTVYLQMNNLKPED | |
| | TAVYYCASGSYYGCTVDDYDFWGQGTQVTVSS | |
| Clone 3A8-hu08 VHH | EVQLQESGGGLVQAGGSLRLSCAASGFTFDEYAIGWVRQAPGKGL | 85 |
| | EGVSCLRVSDGRTYYPDSVKGRFTISRDNSKNTVYLQMNNLKPED | |
| | TAVYYCASGSYYGCTVDDYDFWGQGTTVTVSS | |
| Clone 3A8-hu09 VHH | QVQLQESGGGLVQAGGSLRLSCAASGFTFDEYAIGWFRQAPGKGL | 86 |
| | EGVSCLRVSDGRTYYPDSVKGRFAISSDNAKNTVYLQMNNLKPED | |
| | TAVYYCASGSYYGCTVDDYDFWGQGTQVTVSS | |
| Clone 3A8-hu10 VHH | EVQLQESGGGLVQPGGSLRLSCAASGFTFDEYAIGWFRQAPGKGL | 87 |
| | EGVSCLRVSDGRTYYPDSVKGRFTISRDNAKNTVYLQMNNLKPED | |
| | TAVYYCASGSYYGCTVDDYDFWGQGTTVTVSS | |
| Clone 3A8-hull VHH | EVQLQESGGGLVQAGGSLRLSCAASGFTFDEYAIGWFRQAPGKGL | 88 |
| | EGVSCLRVSDGRTYYPDSVKGRFTISRDNSKNTVYLQMNNLKPED | |
| | TAVYYCASGSYYGCTVDDYDFWGQGTQVTVSS | |
| Clone 3A8-hu12 VHH | EVQLQESGGGLVQAGGSLRLSCAASGFTFDEYAIGWFRQAPGKGL | 89 |
| | EGVSCLRVSDGRTYYPDSVKGRFTISRDNSKNTVYLQMNNLKPED | |
| | TAVYYCASGSYYGCTVDDYDFWGQGTTVTVSS | |
| Clone 3A8-hu13 VHH | QVQLQESGGGLVQAGGSLRLSCAASGFTFDEYAIGWVRQAPGKGR | 90 |
| | EGVSCLRVSDGRTYYPDSVKGRFAISSDNAKNTVYLQMNNLKPED | |
| | TAVYYCASGSYYGCTVDDYDFWGQGTQVTVSS | |
| Clone 3A8-hu14 VHH | EVQLQESGGGLVQPGGSLRLSCAASGFTFDEYAIGWVRQAPGKGR | 91 |
| | EGVSCLRVSDGRTYYPDSVKGRFTISRDNAKNTVYLQMNNLKPED | |
| | TAVYYCASGSYYGCTVDDYDFWGQGTTVTVSS | |
| Clone 3A8-hu15 VHH | EVQLQESGGGLVQAGGSLRLSCAASGFTFDEYAIGWVRQAPGKGR | 92 |
| | EGVSCLRVSDGRTYYPDSVKGRFTISRDNSKNTVYLQMNNLKPED | |
| | TAVYYCASGSYYGCTVDDYDFWGQGTQVTVSS | |
| Clone 3A8-hu16 VHH | EVQLQESGGGLVQAGGSLRLSCAASGFTFDEYAIGWVRQAPGKGR | 93 |
| | EGVSCLRVSDGRTYYPDSVKGRFTISRDNSKNTVYLQMNNLKPED | |
| | TAVYYCASGSYYGCTVDDYDFWGQGTTVTVSS | |
| Clone 3A8-hu17 VHH | QVQLQESGGGLVQAGGSLRLSCAASGFTFDEYAIGWFRQAPGKGR | 94 |
| | EGVSCLRVSDGRTYYPDSVKGRFAISSDNAKNTVYLQMNNLKPED | |
| | TAVYYCASGSYYGCTVDDYDFWGQGTQVTVSS | |
| Clone 3A8-hu18 VHH | EVQLQESGGGLVQPGGSLRLSCAASGFTFDEYAIGWFRQAPGKGR | 95 |
| | EGVSCLRVSDGRTYYPDSVKGRFTISRDNAKNTVYLQMNNLKPED | |
| | TAVYYCASGSYYGCTVDDYDFWGQGTTVTVSS | |
| Clone 3A8-hu19 VHH | EVQLQESGGGLVQAGGSLRLSCAASGFTFDEYAIGWFRQAPGKGR | 96 |
| | EGVSCLRVSDGRTYYPDSVKGRFTISRDNSKNTVYLQMNNLKPED | |
| | TAVYYCASGSYYGCTVDDYDFWGQGTQVTVSS | |
| Clone 3A8-hu20 VHH | EVQLQESGGGLVQAGGSLRLSCAASGFTFDEYAIGWFRQAPGKGR | 97 |
| | EGVSCLRVSDGRTYYPDSVKGRFTISRDNSKNTVYLQMNNLKPED | |
| | TAVYYCASGSYYGCTVDDYDFWGQGTTVTVSS | |
| Clone 3A8-hu21 VHH | QVQLQESGGGLVQAGGSLRLSCAASGFTFDEYAIGWVRQAPGKGL | 98 |
| | EWVSCLRVSDGRTYYPDSVKGRFAISSDNAKNTVYLQMNNLKPED | |
| | TAVYYCASGSYYGCTVDDYDFWGQGTQVTVSS | |
| Clone 3A8-hu22 VHH | EVQLQESGGGLVQPGGSLRLSCAASGFTFDEYAIGWVRQAPGKGL | 99 |
| | EWVSCLRVSDGRTYYPDSVKGRFTISRDNAKNTVYLQMNNLKPED | |
| | TAVYYCASGSYYGCTVDDYDFWGQGTTVTVSS | |
| Clone 3A8-hu23 VHH | EVQLQESGGGLVQAGGSLRLSCAASGFTFDEYAIGWVRQAPGKGL | 100 |
| | EWVSCLRVSDGRTYYPDSVKGRFTISRDNSKNTVYLQMNNLKPED | |
| | TAVYYCASGSYYGCTVDDYDFWGQGTQVTVSS | |
| Clone 3A8-hu24 VHH | EVQLQESGGGLVQAGGSLRLSCAASGFTFDEYAIGWVRQAPGKGL | 101 |
| | EWVSCLRVSDGRTYYPDSVKGRFTISRDNSKNTVYLQMNNLKPED | |
| | TAVYYCASGSYYGCTVDDYDFWGQGTTVTVSS | |
| Clone 3A8-hu25 VHH | QVQLQESGGGLVQAGGSLRLSCAASGFTFDEYAIGWFRQAPGKGL | 102 |
| | EWVSCLRVSDGRTYYPDSVKGRFAISSDNAKNTVYLQMNNLKPED | |
| | TAVYYCASGSYYGCTVDDYDFWGQGTQVTVSS | |
| Clone 3A8-hu26 VHH | EVQLQESGGGLVQPGGSLRLSCAASGFTFDEYAIGWFRQAPGKGL | 103 |
| | EWVSCLRVSDGRTYYPDSVKGRFTISRDNAKNTVYLQMNNLKPED | |
| | TAVYYCASGSYYGCTVDDYDFWGQGTTVTVSS | |
| Clone 3A8-hu27 VHH | EVQLQESGGGLVQAGGSLRLSCAASGFTFDEYAIGWFRQAPGKGL | 104 |
| | EWVSCLRVSDGRTYYPDSVKGRFTISRDNSKNTVYLQMNNLKPED | |
| | TAVYYCASGSYYGCTVDDYDFWGQGTQVTVSS | |
| Clone 3A8-hu28 VHH | EVQLQESGGGLVQAGGSLRLSCAASGFTFDEYAIGWFRQAPGKGL | 105 |
| | EWVSCLRVSDGRTYYPDSVKGRFTISRDNSKNTVYLQMNNLKPED | |
| | TAVYYCASGSYYGCTVDDYDFWGQGTTVTVSS | |

### 1. Binding Affinity of Anti-CD8 Antibodies

In some embodiments, the anti-CD8 antibodies provided herein have an equilibrium dissociation constant (K_{D}) for binding to CD8 of <100 nM, <10 nM, <1 nM, <0.1 nM, <0.01 nM, or <0.001 nM (e.g., 10⁻⁸ M or less, from 10⁻⁸ M to 10⁻¹³ M, e.g., from 10⁻⁹ M to 10⁻¹³ M). In some embodiments, the binding affinity is measured by equilibrium dissociation constant (K_{D}) to the hu-CD8 ECD polypeptide of SEQ ID NO: 1. In some embodiments, the anti-CD8 antibody is capable of binding with an affinity of an equilibrium dissociation constant (K_{D}) for binding to CD8 of <100 nM, <10 nM, <1 nM, <0.1 nM, <0.01 nM, or <0.001 nM (e.g., 10⁻⁸ M or less, from 10⁻⁸ M to 10⁻¹³ M, e.g., from 10⁻⁹ M to 10⁻¹³ M) to the hu-CD8 ECD SEQ ID NO: 1.

It is contemplated that the various anti-CD8 antibodies generated as disclosed herein include antibodies capable of high-affinity binding to cyno-CD8, and/or to both hu-CD8 and cyno-CD8. In some embodiments, the anti-CD8 antibodies of the present disclosure bind to cyno-CD8 with a binding affinity of 1×10⁻⁸ M or less, 1×10⁻⁹ M or less, 1×10⁻¹⁰ M or less, or 1×10⁻¹¹ M or less.

Generally, the binding affinity of a ligand to its receptor can be determined using any of a variety of assays and expressed in terms of a variety of quantitative values. Specific CD8 binding assays useful in determining affinity of the antibodies are disclosed in the Examples herein. Additionally, antigen binding assays are known in the art and can be used herein including without limitation any direct or competitive binding assays using techniques such as western blots, radioimmunoassays, enzyme-linked immunoabsorbent assay (ELISA), “sandwich” immunoassays, surface plasmon resonance based assay (such as the BIAcore assay as described in WO2005/012359), immunoprecipitation assays, fluorescent immunoassays, protein A immunoassays, flow cytometric and fluorescence activated cell sorting (FACS) assays, and the like. Accordingly, in some embodiments, the binding affinity is expressed as K_{D} values and reflects intrinsic binding affinity (e.g., with minimized avidity effects). The anti-CD8 antibodies of the present disclosure exhibit strong binding affinities for the extracellular domains of the two CD8 isoforms, CD8α (or CD8A) and CD8B (or CD8B). These two isoforms CD8 ECD can occur as homodimer (e.g., CD8A/CD8A) or a heterodimer (e.g., CD8A/CD8B) on CD8+ cells. The anti-CD8 antibodies of the present disclosure may compete with antibodies having lower affinity for the same or overlapping epitopes of CD8A and/or CD8B in homodimeric or heterodimeric forms.

In some embodiments, the anti-CD8 antibodies provided herein specifically target and bind to CD8 expressed on CD8+ T cells, without decreasing, inhibiting, or blocking the immune regulatory effects mediated by CD8+ T-cells, including the activation of CD8+ T cells in response to a specific MHC antigen binding. In some embodiments, the anti-CD8 antibodies provided herein specifically target and bind to CD8 expressed on CD8+ T cells, and inhibit the immune regulatory and/or immune signaling pathways mediated by an activated CD8+ cell binding. The effects of antibody binding on the CD8 mediated immune response can be assayed in vitro using known cell-based assays including the cell-based assays described in the Examples of the present disclosure. Accordingly, in some embodiments, the anti-CD8 antibodies of the present disclosure are characterized by one or more of following functional properties based on the ability to alter activation CD8+ T cells and the associated immune response.

In at least one embodiment, the anti-CD8 antibody of the present disclosure blocks binding to hu-CD8A ECD (SEQ ID NO: 1) measured by ELISA with an IC₅₀ of 10 nM or less, 7 nM or less, 5 nM or less, 2 nM or less, 1 nM or less, 0.5 nM or less, or 0.25 nM or less.

In at least one embodiment, the anti-CD8 antibody of the present disclosure blocks binding to hu-CD8A ECD expressed on a cell with an IC₅₀ of 2.5 nM or less, 1 nM or less, 0.5 nM or less, or 0.25 nM or less; optionally, wherein the cell is a HEKBlue cell stably expressing CD8A ECD.

In at least one embodiment, the anti-CD8 antibody of the present disclosure blocks binding to hu-CD8A ECD expressed on a human T cell with an IC₅₀ of 5 nM or less, 2.5 nM or less, 1 nM or less, 0.5 nM or less, or 0.25 nM or less; optionally, wherein the cell is a human CD8+ T-cell.

### 2. Antibody Fragments

As described elsewhere herein, the anti-CD8 antibodies of the present disclosure comprise a single heavy chain variable domain (VH), each with three complementarity determining regions (CDR-H1, CDR-H2, and CDR-H3), and can also be considered antibody fragments. The binding determinants of these anti-CD8 antibody fragments can be formatted in a variety of antibody fragment types including, but not limited to, Fab, Fab′, Fab′-SH, F(ab′)₂, Fv, scFv fragments, monovalent, single domain antibody (e.g., VHH), one-armed or single-arm antibody, and other fragments described herein and known in the art. Accordingly, in some embodiments of the anti-CD8 antibodies of the present disclosure, the antibody is an antibody fragment selected from the group consisting of F(ab′)₂, Fab′, Fab, Fv, single domain antibody, VHH, single-arm antibody, and scFv.

For a review of various antibody fragments, see e.g., Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO93/16185; and U.S. Pat. Nos. 5,571,894 and 5,587,458. For a description of Fab and F(ab′)₂ fragments comprising salvage receptor binding epitope residues and having increased in vivo half-life, see U.S. Pat. No. 5,869,046. Other monovalent antibody forms are described in, e.g., WO2007/048037, WO2008/145137, WO2008/145138, and WO2007/059782. Monovalent, single-armed antibodies are described, e.g., in WO2005/063816. Diabodies are antibody fragments with two antigen-binding sites that may be bivalent or bispecific (see e.g., EP0404097; WO93/01161; Hudson et al., Nat. Med. 9:129-134 (2003); and Hollinger et al., Proc. Natl. Acad. Sci. USA 90:6444-6448 (1993)).

In some embodiments, the antibody fragments are single-domain antibodies which comprise all or a portion of the heavy chain variable domain or all or a portion of the light chain variable domain of an antibody. In some embodiments, a single-domain antibody is a human single-domain antibody (Domantis, Inc., Waltham, MA; see, e.g., U.S. Pat. No. 6,248,516).

Antibody fragments can be made by various techniques, including but not limited to proteolytic digestion of an intact antibody as well as production by recombinant host cells (e.g., E. coli or phage).

### 3. Chimeric and Humanized Antibodies

In some embodiments, the anti-CD8 antibody of the present disclosure can be a chimeric antibody. (See e.g., chimeric antibodies as described in U.S. Pat. No. 4,816,567; and Morrison et al., Proc. Natl. Acad. Sci. USA, 81:6851-6855 (1984)). In one embodiment, a chimeric antibody comprises a non-human variable region (e.g., a variable region derived from a mouse, rat, hamster, rabbit, or non-human primate, such as a monkey) and a human constant region. In some embodiments, a chimeric antibody is a “class switched: antibody in which the class or subclass has been changed from that of the parent antibody. It is contemplated that chimeric antibodies can include antigen-binding fragments thereof.

In some embodiments, the anti-CD8 antibody of the present disclosure is a humanized antibody. Typically, a non-human antibody is humanized to reduce immunogenicity to humans, while retaining the specificity and affinity of the parental non-human antibody. Generally, a humanized antibody comprises one or more variable domains in which HVRs, CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the CDR residues are derived) to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al, Methods 36:25-34 (2005) (describing SDR (a-HVR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol, 151:2623 (1993)); human mature (somatically mutated) framework regions or human germline framework regions (see, e.g., Almagro and Fransson, Front. Biosci. 13: 1619-1633 (2008)); and framework regions derived from screening FR libraries (see, e.g., Baca et al., J. Biol. Chem. 272: 10678-10684 (1997) and Rosok et al., J. Biol. Chem. 271: 22611-22618 (1996)).

### 4. Human Antibodies

In some embodiments, the anti-CD8 antibody of the present disclosure can be a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20: 450-459 (2008). Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23: 1117-1125 (2005). See also, e.g., XENOMOUSE™ technology in U.S. Pat. Nos. 6,075,181 and 6,150,584; HUMAB® technology in U.S. Pat. No. 5,770,429; K-M MOUSE® technology in U.S. Pat. No. 7,041,870; and VELOCIMOUSE® technology in U.S. Pat. Appl. Pub. No. US 2007/0061900). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. See, e.g., Kozbor J. Immunol, 133:3001 (1984); Brodeur et al., Monoclonal Antibody Production Techniques and Applications, pp. 51-63 (Marcel Dekker, Inc., New York, 1987); and Boerner et al., J. Immunol., 147:86 (1991). Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines). Human hybridoma technology (Trioma technology) is also described in Vollmers and Brandlein, Histology and Histopathology, 20 (3): 927-937 (2005) and Vollmers and Brandlein, Methods and Findings in Experimental and Clinical Pharmacology, 27 (3): 185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### 5. Library-Derived Antibodies

In some embodiments, the anti-CD8 antibody of the present disclosure may be isolated by screening combinatorial libraries for antibodies with the desired activity or activities. For example, a variety of methods are known in the art for generating phage display libraries and screening such libraries for antibodies possessing the desired binding characteristics. Other methods for producing such library-derived antibodies can be found in e.g., Hoogenboom et al., Methods in Molecular Biology 178:1-37 (O'Brien et al., ed., Human Press, Totowa, NJ, 2001); McCafferty et al., Nature 348:552-554; Clackson et al., Nature 352:624-628 (1991); Marks et al., J. Mol. Biol. 222:581-597 (1992); Marks and Bradbury, m Methods in Molecular Biology 248:161-175 (Lo, ed., Human Press, Totowa, NJ, 2003); Sidhu et al., J. Mol. Biol. 338 (2): 299-310 (2004); Lee et al., J. Mol. Biol. 340 (5): 1073-1093 (2004); Fellouse, Proc. Natl. Acad. Sci. USA 101 (34): 12467-12472 (2004); and Lee et al., J. Immunol. Methods 284 (1-2): 119-132 (2004).

### 6. Multispecific Antibodies

In some embodiments, the anti-CD8 antibody of the present disclosure is a multispecific antibody, e.g., a bispecific antibody. In some embodiments, the multispecific antibody is a monoclonal antibody having at least two different binding sites, each with a binding specificity for a different antigen, at least one of which specifically binds to CD8.

In some embodiments, the multispecific antibody is a bispecific antibody comprising a specificity for CD8 and a specificity for another antigen that mediates immune regulation, immune signaling, and/or is expressed on a cancer or tumor cell. In some embodiments of the bispecific antibody, the other specificity is for an antigen that is an immune checkpoint molecule, such as PD1, CTLA4, and LAG3.

In some embodiments, at least one of binding sites specifically binds a cytotoxic agent. In exemplary embodiments, an anti-CD8 antibody of the present disclosure is a bispecific antibody and can be used to localize a cytotoxic agent to CD8+ cells.

Techniques for making multispecific antibodies include, but are not limited to, recombinant co-expression of two immunoglobulin heavy chain-light chain pairs having different specificities (see e.g., Milstein and Cuello, Nature 305:537 (1983), WO 93/08829, and Traunecker et al., EMBOJ. 10:3655 (1991)). “Knob-in-hole” engineering can also be used to generate bispecific antibodies useful with the anti-CD8 antibodies of the present disclosure. Techniques for knob-in-hole engineering are known in the art and described in e.g., U.S. Pat. No. 5,731,168.

Multispecific antibodies can also be made by engineering “electrostatic steering” effects that favor formation of Fc-heterodimeric antibody molecules rather than homodimers (WO 2009/089004A1); cross-linking two or more antibodies or fragments (see, e.g., U.S. Pat. No. 4,676,980, and Brennan et al., Science, 229:81 (1985)); using leucine zippers to produce bispecific antibodies (see, e.g., Kostelny et al., J. Immunol, 148 (5): 1547-1553 (1992)); using “diabody” technology for making bispecific antibody fragments (see, e.g., Hollinger et al., Proc. Natl. Acad. Sci. USA, 90:6444-6448 (1993)); using single-chain Fv (scFv) dimers (see, e.g. Gruber et al., J. Immunol, 152:5368 (1994)); or tri-specific antibodies (see e.g., Tutt et al., J. Immunol. 147:60 (1991).

### 7. Antibody Variants

In some embodiments, variants of the anti-CD8 antibody of the present disclosure are also contemplated. For example, antibodies with improved binding affinity and/or other biological properties of the antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristic of CD8 antigen binding.

### A. Substitution, Insertion, and Deletion Variants

In some embodiments, anti-CD8 antibody variants having one or more amino acid substitutions in addition to those described herein are provided. Sites for mutagenesis can include the HVRs and FRs. Typical “conservative” amino acid substitutions and/or substitutions based on common side-chain class or properties are well-known in the art and can be used in the embodiments of the present disclosure. The present disclosure also contemplates variants based on non-conservative amino acid substitutions in which a member of one of amino acid side chain class is exchanged for an amino acid from another class.

Amino acid side chains are typically grouped according to the following classes or common properties: (1) hydrophobic: Met, Ala, Val, Leu, Ile, Norleucine; (2) neutral hydrophilic: Cys, Ser, Thr, Asn, Gln; (3) acidic: Asp, Glu; (4) basic: His, Lys, Arg; (5) chain orientation influencing: Gly, Pro; and (6) aromatic: Trp, Tyr, Phe.

Techniques are well-known in the art for amino acid substitution into an antibody and subsequent screening for desired function, e.g., retained/improved antigen binding, decreased immunogenicity, or improved ADCC or CDC.

Amino acid substitution variants can include substituting one or more hypervariable region residues of a parent antibody (e.g., a humanized or human antibody). Generally, the resulting variant(s) selected for further study will have modifications in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques. Briefly, one or more CDR residues are mutated and the variant antibodies displayed on phage and screened for a particular biological activity (e.g., binding affinity).

A useful method for identifying residues or regions of an antibody that may be targeted for mutagenesis is “alanine scanning mutagenesis” (see e.g., Cunningham and Wells (1989) Science, 244: 1081-1085). In this method, a residue or group of target residues (e.g., charged residues such as Arg, Asp, His, Lys, and Glu) are identified and replaced by a neutral or negatively charged amino acid (e.g., Ala or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen can be determined. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intra-sequence insertions of single or multiple amino acid residues. Examples of terminal insertions include an antibody with an N-terminal methionyl residue. Other insertional variants of the antibody molecule include the fusion to the N- or C-terminus of the antibody to an enzyme or a polypeptide which increases the serum half-life of the antibody.

Substitutions can be made in HVRs to improve antibody affinity. Such alterations may be made in “hotspots,” i.e., residues encoded by codons that undergo mutation at high frequency during the somatic maturation process (see, e.g., Chowdhury, Methods Mol. Biol. 207:179-196 (2008)) with the resulting variant V_{H} or V_{L} being tested for binding affinity. In some embodiments, affinity maturation can be carried out by constructing and reselecting from secondary libraries (see e.g., in Hoogenboom et al., Methods in Molecular Biology 178:1-37 (O'Brien et al., ed., Human Press, Totowa, NJ, (2001).) Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. HVR-H3 and HVR-L3 in particular are often targeted.

In some embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots.” In some embodiments of the variant V_{H} and V_{L} sequences provided above, each HVR either is unaltered, or contains no more than one, two or three amino acid substitutions.

### B. Glycosylation Variants

In some embodiments, the anti-CD8 antibody of the present disclosure is altered to increase or decrease the extent to which the antibody is glycosylated. Addition or deletion of glycosylation sites to an antibody can be carried out by altering the amino acid sequence such that one or more glycosylation sites can be created or removed.

In embodiments where the antibody comprises an Fc region, the carbohydrate attached to the Fc region can be altered. Typically, native antibodies produced by mammalian cells comprise a branched, biantennary oligosaccharide attached by an N-linkage to the asparagine at about position 297 (“N297”) of the CH2 domain of the Fc region (see, e.g., Wright et al. TIBTECH 15:26-32 (1997)). The oligosaccharide may include various carbohydrates, such as mannose, N-acetyl glucosamine (GlcNAc), galactose, and sialic acid, as well as, a fucose attached to a GlcNAc in the “stem” of the bi-antennary oligosaccharide structure. In some embodiments, the modifications of the oligosaccharide of an Fc region of an antibody can create a variant with certain improved properties.

In some embodiments, the anti-CD8 antibody of the present disclosure can be a variant of a parent antibody, wherein the variant comprises a carbohydrate structure that lacks fucose attached (directly or indirectly) to an Fc region. For example, the amount of fucose in such antibody may be from about 1% to about 80%, from about 1% to about 65%, from about 5% to about 65%, or from about 20% to about 40%. The amount of fucose can be determined by calculating the average amount of fucose within the sugar chain at N297, relative to the sum of all glyco-structures attached to Asn 297 (e.g., complex, hybrid and high mannose structures) as measured by MALDI-TOF mass spectrometry (see e.g., WO 2008/077546). N297 refers to the asparagine residue located at about position 297 in the Fc region (Bu numbering of Fc region residues); however, N297 may also be located about +3 amino acids upstream or downstream of position 297, i.e., between positions 294 and 300, due to minor sequence variations in antibodies.

In some embodiments, the fucosylation variants can have improved ADCC function. See, e.g., US Patent Publication Nos. US 2003/0157108, or US 2004/0093621. Examples of “defucosylated” or “fucose-deficient” antibodies and associated methods for preparing them are disclosed in e.g., US2003/0157108; US2003/0115614; US2002/0164328; US2004/0093621; US2004/0132140; US2004/0110704; US2004/0110282; US2004/0109865; WO2000/61739; WO2001/29246; WO2003/085119; WO2003/084570; WO2005/035586; WO2005/035778; WO2005/053742; WO2002/031140; Okazaki et al. J. Mol. Biol. 336:1239-1249 (2004); Yamane-Ohnuki et al. Biotech. Bioeng. 87:614 (2004).

Cell lines useful for producing defucosylated antibodies include Led 3 CHO cells deficient in protein fucosylation (see e.g., Ripka et al. Arch. Biochem. Biophys. 249: 533-545 (1986); US2003/0157108, and WO2004/056312), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94 (4): 680-688 (2006); and WO2003/085107).

### C. Fc Region Variants

In some embodiments, an anti-CD8 antibody of the present disclosure can comprise one or more amino acid modifications in the Fe region (i.e., an Fc region variant). The Fc region variant may comprise a human Fc region sequence (e.g., a human IgG1, IgG2, IgG3, or IgG4 Fc region) comprising an amino acid substitution at one or more amino acid residue positions. A wide range of Fc region variants known in the art that are useful with the anti-CD8 antibodies of the present disclosure are described below.

In some embodiments, the anti-CD8 antibody can be an Fc region variant which has altered effector function. In some embodiments, the antibody with altered effector function can possess some (but not all of) the effector functions, decreased effector function, or none of the effector functions (e.g., effectorless) of the parent antibody. Effectorless Fc region variants can be more desirable for certain applications where effector function (such as ADCC) is unnecessary or deleterious, and/or in vivo half-life of the antibody is important.

Fc region variant antibodies with reduced effector function, or which are effectorless, can include an amino acid substitution at one or more of the following Fc region positions: 238, 265, 269, 270, 297, 327 and 329. (see, e.g., U.S. Pat. No. 6,737,056). Such Fc region variants can include amino acid substitutions at two or more of positions 265, 269, 270, 297 and 327. Such Fc region variants can also include substitutions of both residues 265 and 297 to alanine (see e.g., U.S. Pat. No. 7,332,581). In some embodiments, the anti-CD8 antibodies of the present disclosure are effectorless Fc region variants. In some embodiments, the effectorless Fc region variants of the anti-CD8 antibodies comprise one or more amino acid substitutions selected from N297G (see e.g., Shields, R. et al., “High Resolution Mapping of the Binding Site on Human IgG1 for FcγRI, FcγRII, FcγRIII and FcRn and Design of IgG1 Variants with Improved Binding to the FcγR*”, Journal of Biological Chemistry, 276 (9): 6591-6604 (2001)); N297A (see e.g., Friend, P. J. et al., “Phase I Study of an Engineered Aglycosylated Humanized CD3 Antibody in Renal Transplant Rejection”, Transplantation, 68 (11): 1632-7 (1999)); P331S/K322A (see e.g., Tawara, T. et al., “Complement Activation Plays a Key Role in Antibody-Induced Infusion Toxicity in Monkeys and Rats”, J Immunol, 180 (4): 2294-8 (2008)); S228P/L235E (see e.g., Newman, R. et al., “Modification of the Fc Region of a Primatized IgG Antibody to Human CD4 Retains Its Ability to Modulate CD4 Receptors but Does Not Deplete CD4(+) T Cells iN Chimpanzees”, Clin Immunol, 98 (2): 164-74 (2001); or L234A/L235A/P329G (also referred to as “LALAPG”) (see e.g., Schlothauer, T. et al., “Novel human IgG1 and IgG4 Fc-engineered antibodies with completely abolished immune effector functions”, Protein Eng. Des. Sel., 29 (10): 457-466 (2016); Lo, M. et al., “Effector-attenuating Substitutions That Maintain Antibody Stability and Reduce Toxicity in Mice”, Journal of Biological Chemistry, 292 (9): 3900-3908 (2017). In other embodiments, the effectorless Fe region variants of the anti-CD8 antibodies comprise the amino acid substitutions L234A/L235A (“LALA”) (Woodle, E. Steve et al., Transplantation, 68 (5): 608-616 (1999)).

Accordingly, in some embodiments, the effectorless Fc region variants of the anti-CD8 antibodies comprise one or more amino acid substitutions selected from N297A or N297G. In some embodiments, the effectorless Fc region variants of the anti-CD8 antibodies comprise the pair of amino acid substitutions P331S/K322A. In other embodiments, the effectorless Fc region variants of the anti-CD8 antibodies comprise the amino acid substitutions L234A/L235A (LALA) or L234A/L235A/P329G (LALAPG). In some embodiments, wherein the anti-CD8 is of isotype IgG2 or IgG4, the anti-CD8 antibody comprises the amino acid substitutions S228P and/or L235E.

Fc region variants having improved or diminished binding to FcRs are disclosed in e.g., U.S. Pat. No. 6,737,056; WO 2004/056312; and Shields et al., J. Biol. Chem. 9 (2): 6591-6604 (2001). Fc region variants having improved ADCC can comprise one or more amino acid substitutions at e.g., positions 298, 333, and/or 334 of the Fc region (based on EU numbering). Fc region variants having altered (i.e., either improved or diminished) C1q binding and/or Complement Dependent Cytotoxicity (CDC), as described in e.g., U.S. Pat. No. 6,194,551, WO99/51642, and Idusogie et al., J. Immunol. 164:4178-4184 (2000). Fc region variants with increased half-lives and improved binding to the neonatal Fe receptor (FcRn) are disclosed in e.g., US2005/0014934A1 (Hinton et al.). Such Fc region variants comprise amino acid substitutions at one or more of positions: 238, 256, 265, 272, 286, 303, 305, 307, 311, 312, 317, 340, 356, 360, 362, 376, 378, 380, 382, 413, 424, and 434. Other Fc region variants with increased half-lives include the set of YTE mutations at positions 252, 254, and 256 (i.e., M252Y/S254T/T256E) described in e.g., U.S. Pat. No. 7,658,921B2 (Dall'Acqua et al.). Other examples of Fc region variants can be found in e.g., U.S. Pat. Nos. 5,648,260 and 5,624,821; and WO94/29351.

Generally, in vitro and/or in vivo cytotoxicity assays can be carried out to confirm the reduction/depletion of CDC and/or ADCC activities in an Fc region variant. For example, Fc receptor (FcR) binding assays can be conducted to ensure that the antibody lacks FcγR binding (hence likely lacking ADCC activity) but retains FcRn binding ability. The primary cells for mediating ADCC, NK cells express FcγRIII only, whereas monocytes express FcγRI, FcγRII, and FcγRIII. Non-limiting examples of in vitro assays to assess ADCC activity of a molecule of interest is described in U.S. Pat. No. 5,500,362 (see, e.g. Hellstrom, et al., Proc. Nat'l Acad. Sci. USA 83:7059-7063 (1986)) and Hellstrom, et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assay methods may be employed (see, for example, ACTI™ nonradioactive cytotoxicity assay for flow cytometry (CellTechnology, Inc. Mountain View, CA; and CytoTox96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). Clq binding assays may also be carried out to confirm that the antibody is unable to bind C1q and hence lacks CDC activity. See, e.g., C1q and C3c binding ELISA in WO2006/029879 and WO2005/100402. To assess complement activation, a CDC assay may be performed (see, e.g., Gazzano-Santoro et al., J. Immunol. Methods 202:163 (1996); Cragg, M. S. et al., Blood 101:1045-1052 (2003); and Cragg, M. S. and M. J. Glennie, SW 103:2738-2743 (2004)). FcRn binding and in vivo clearance/half-life determinations can be performed using methods known in the art (see, e.g., Petkova, et al., Intl. Immunol. 18 (12): 1759-1769 (2006)).

### D. Cysteine Engineered Antibody Variants

In some embodiments, it is contemplated that the anti-CD8 antibody described herein can be substituted at specific non-HVR positions with cysteine residues so as to create reactive thiol groups. Such engineered “thioMAbs” can be used to conjugate the antibody to e.g., drug moieties or linker-drug moieties and thereby create immunoconjugates, as described elsewhere herein. Cysteine engineered antibodies can be generated as described in e.g., U.S. Pat. No. 7,521,541. In some embodiments, any one or more of the following antibody residues can be substituted with cysteine: V205 (Kabat numbering) of the light chain; A118 (EU numbering) of the heavy chain; and S400 (EU numbering) of the heavy chain Fc region.

### E. Antibody Derivatives

In some embodiments, the anti-CD8 antibody of the present disclosure may be further modified (i.e., derivatized) with non-proteinaceous moieties. Non-proteinaceous moieties suitable for derivatization of the antibody include, but are not limited to, water soluble polymers, such as: polyethylene glycol (PEG), copolymers of ethylene glycol and propylene glycol, carboxy-methylcellulose, dextran, polyvinyl alcohol, polyvinyl pyrrolidone, poly-1,3-dioxolane, poly-1,3,6-trioxane, ethylene/maleic anhydride copolymer, poly-amino acid homo-polymers or random co-polymers, and dextran or poly(n-vinyl pyrrolidone) polyethylene glycol, propropylene glycol homo-polymers, polypropylene oxide/ethylene oxide co-polymers, polyoxy-ethylated polyols (e.g., glycerol), polyvinyl alcohol, and mixtures thereof. In some embodiments, modification of the antibody can be carried out using methoxy-polyethylene glycol propionaldehyde. The polymers may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody, e.g., whether the antibody derivative will be used in a therapy under defined conditions.

### 8. Immunoconjugates

In some embodiments, the anti-CD8 antibody of the present disclosure can also be an immunoconjugate, wherein the immunoconjugate comprises an anti-CD8 antibody conjugated to one or more cytotoxic agents. Suitable cytotoxic agents contemplated by the present disclosure include chemotherapeutic agents, drugs, growth inhibitory agents, toxins (e.g., protein toxins, enzymatically active toxins of bacterial, fungal, plant, or animal origin, or fragments thereof), or radioactive isotopes.

In some embodiments, the immunoconjugate is an antibody-drug conjugate (ADC) in which an anti-CD8 antibody, as described herein, is conjugated to one or more drugs.

In some embodiments, an immunoconjugate of the present disclosure comprises an anti-CD8 antibody as described herein conjugated to a drug or therapeutic agent for the treatment of a CD8-mediated disease or condition.

In some embodiments, an anti-CD8 antibody as described herein can be conjugated to an enzymatically active toxin or a fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins, Momordica charantia inhibitor, curcin, crotin, Sapaonaria officinalis inhibitor, gelonin, mitogellin, restrictocin, phenomycin, enomycin, and the tricothecenes.

In some embodiments, an immunoconjugate of the present disclosure comprises an anti-CD8 antibody as described herein conjugated to a radioactive isotope (i.e., a radioconjugate). A variety of radioactive isotopes are available for the production of such radioconjugates. Examples include, but are not limited to, ⁶⁴Cu, ⁸⁹Zr, ²¹¹At, ¹³¹I, ¹²⁵I, ⁹⁰Y, ¹⁸⁶Re, ¹⁸⁸Re, ¹⁵³Sm, ²¹²Bi, ³²P, ²¹²Pb, and radioactive isotopes of Lu. In some embodiments, a radioactive isotope can be or comprise ¹⁸F, FDG. In some embodiments, the immunoconjugate may comprise a radioisotope for scintigraphic detection, or a spin label for NMR detection or MRI. Suitable radioisotopes or spin labels can include, as ¹²³I, ¹³¹I, ¹¹¹In, ¹³C, ¹⁹F, ¹⁵N, ¹⁷O, various isotopes of Gd, Mn, and Fe.

Immunoconjugates of an anti-CD8 antibody and a cytotoxic agent, can be made using a variety of well-known bifunctional reagents and chemistries suitable for conjugating to proteins. Such reagents include but are not limited to: N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (e.g., dimethyl adipimidate HQ), active esters (e.g., disuccinimidyl suberate), aldehydes (e.g., glutaraldehyde), bis-azido compounds (e.g., bis-(p-azidobenzoyl)-hexanediamine), bis-diazonium derivatives (e.g., bis-(p-diazoniumbenzoyl)-ethylenediamine), diisocyanates (e.g., toluene-2,6-diisocyanate), and bis-active fluorine compounds (e.g., 1,5-difluoro-2,4-dinitrobenzene).

Reagents for preparing immunoconjugates of the present disclosure can also include commercially available “cross-linking” reagents such as: BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) (see e.g., Pierce Biotechnology, Inc., Rockford, IL., U.S.A).

### 9. Synthetic Antibodies

In some embodiments, the anti-CD8 antibody of the present disclosure can be a synthetic antibody comprising a set of CDRs or HVRs from an anti-CD8 immunoglobulin (e.g., CDR-H1, etc.) grafted onto a scaffold or framework other than an immunoglobulin scaffold or framework, such as an alternative protein scaffold, or an artificial polymer scaffold.

Exemplary alternative protein scaffolds contemplated for preparation of synthetic antibodies of the present disclosure can include, but are not limited to: fibronectin, neocarzinostatin CBM4-2, lipocalins, T-cell receptor, protein-A domain (protein Z), Im9, TPR proteins, zinc finger domains, p VIII, avian pancreatic polypeptide, GCN4, WW domain Src homology domain 3, PDZ domains, TEM-1 beta-lactamase, thioredoxin, staphylococcal nuclease, PHD-fmger domains, CL-2, BPTI, APPI, HPSTI, ecotin, LACI-D1, LDTI, MTI-II, scorpion toxins, insect defensin-A peptide, EETI-II, Min-23, CBD, PBP, cytochrome b-562, Ldl receptor domains, gamma-crystallin, ubiquitin, transferrin, and/or C-type lectin-like domains.

Exemplary artificial polymer (non-protein) scaffolds useful for synthetic antibodies are described in e.g., Fiedler et al., (2014) “Non-Antibody Scaffolds as Alternative Therapeutic Agents,” in Handbook of Therapeutic Antibodies (eds S. Dübel and J. M. Reichert), Wiley-VCH Verlag GmbH & Co.; Gebauer et al., Curr. Opin. Chem. Biol, 13:245-255 (2009); Binz et al, Nat. Biotech., 23 (10): 1257-1268 (2005).

### IV. Recombinant Methods and Compositions

The anti-CD8 antibody of the present disclosure can be produced using recombinant methods and materials well-known in the art of antibody production. In some embodiments, the present disclosure provides an isolated nucleic acid encoding an anti-CD8 antibody. The nucleic acid can encode an amino acid sequence comprising the V_{L} and/or an amino acid sequence comprising the V_{H} of the antibody (e.g., the light and/or heavy chains of the antibody). In some embodiments, one or more vectors (e.g., expression vectors) comprising nucleic acid sequences encoding an anti-CD8 antibody of the present disclosure are provided. In some embodiments, a host cell comprising nucleic acid sequences encoding an anti-CD8 antibody of the present disclosure are provided. In one embodiment, the host cell has been transformed with a vector comprising a nucleic acid that encodes an amino acid sequence comprising the V_{L} of the antibody and an amino acid sequence comprising the V_{H} of the antibody. In another embodiment, the host cell has been transformed with a first vector comprising a nucleic acid that encodes an amino acid sequence comprising the V_{L} of the antibody and a second vector comprising a nucleic acid that encodes an amino acid sequence comprising the V_{H} of the antibody.

In some embodiments of the recombinant methods, the host cell used is a eukaryotic cell, such as a Chinese Hamster Ovary (CHO) cell, or a lymphoid cell (e.g., Y0, NS0, Sp20). In at least one embodiment, a method of making an anti-CD8 antibody is provided, wherein the method comprises culturing a host cell comprising a nucleic acid encoding the antibody, as provided above, under conditions suitable for expression of the antibody, and optionally recovering the antibody from the host cell (or host cell culture medium)

Briefly, recombinant production of an anti-CD8 antibody is carried out by isolating a nucleic acid encoding an antibody (e.g., as described herein) and inserting this nucleic acid into one or more vectors for further cloning and/or expression in a host cell. Such nucleic acids are readily isolated and sequenced using conventional procedures well-known in the art (e.g., by using oligonucleotide probes that are capable of binding specifically to genes encoding the heavy and light chains of the desired antibody). Suitable host cells and culturing methods for cloning or expressing the antibody-encoding vectors are well-known in the art and include prokaryotic or eukaryotic cells. Typically, after expression, the antibody may be isolated from cell paste in a soluble fraction and further purified. In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern (see e.g., Gerngross, Nat. Biotech. 22: 1409-1414 (2004), and Li et al., Nat. Biotech. 24: 210-215 (2006)).

Suitable host cells for the expression of glycosylated anti-CD8 antibodies of the present disclosure can also be derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts (see, e.g., U.S. Pat. Nos. 5,959,177, 6,040,498, 6,420,548, and 7,125,978.

Examples of mammalian host cell lines useful for the production of the anti-CD8 antibodies of the present disclosure include Chinese hamster ovary (CHO) cells, including DHFR-CHO cells (see e.g., Urlaub et al., Proc. Natl. Acad. Sci. USA 77:4216 (1980)); myeloma cell lines such as Y0, NS0 and Sp2/0; monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse Sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TR1 cells (see e.g., in Mather et al., Annals N Y. Acad. Sci. 383:44-68 (1982) and U.S. Pat. No. 6,235,498); Medical Research Council 5 (MRC 5) cells (such as e.g., those available from ATCC and also referred to as CCL-171); and Foreskin 4 (FS-4) cells (see e.g., in Vilcek et al. Ann. N. Y. Acad. Sci. 284: 703-710 (1977), Gardner & Vilcek. J. Gen. Virol. 44: 161-168 (1979), and Pang et al. Proc. Natl. Acad. Sci. U.S.A. 77: 5341-5345 (1980)). For a general review of useful mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

### V. Pharmaceutical Compositions and Formulations of Anti-CD8 Antibodies

The present disclosure also provides pharmaceutical compositions and pharmaceutical formulations comprising an anti-CD8 antibody. In some embodiments, the present disclosure provides a pharmaceutical formulation comprising an anti-CD8 antibody as described herein and a pharmaceutically acceptable carrier. In some embodiments, the anti-CD8 antibody is the sole active agent of the pharmaceutical composition. Such pharmaceutical formulations can be prepared by mixing an anti-CD8 antibody, having the desired degree of purity, with one or more pharmaceutically acceptable carriers. Typically, such antibody formulations can be prepared as an aqueous solution (see e.g., U.S. Pat. No. 6,171,586, and WO2006/044908) or as a lyophilized formulation (see e.g., U.S. Pat. No. 6,267,958).

Pharmaceutically acceptable carriers are generally nontoxic to recipients at the dosages and concentrations employed. A wide range of such pharmaceutically acceptable carriers are well-known in the art (see e.g., Remington's Pharmaceutical Sciences 16th edition, Osol, A. Ed. (1980)). Exemplary pharmaceutically acceptable carriers useful in the formulations of the present disclosure can include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

Pharmaceutically acceptable carriers useful in the formulations of the present disclosure can also include interstitial drug dispersion agents, such as soluble neutral-active hyaluronidase glycoproteins (sHASEGP) (see e.g., US Pat. Publ. Nos. 2005/0260186 and 2006/0104968), such as human soluble PH-20 hyaluronidase glycoproteins (e.g., rHuPH20 or HYLENEX®, Baxter International, Inc.).

It is also contemplated that the formulations disclosed herein may contain active ingredients in addition to the anti-CD8, as necessary for the particular indication being treated in the subject to whom the formulation is administered. Preferably, any additional active ingredient has activity complementary to that of the anti-CD8 antibody activity and the activities do not adversely affect each other.

In some embodiments, the pharmaceutical composition comprises the anti-CD8 antibody and an additional active agent such as, but not limited to, a checkpoint inhibitor. Checkpoint inhibitors useful in such embodiments include, but are not limited to, a second antibody comprising a specificity for an antigen that is an immune checkpoint molecule. In some embodiments, the second antibody comprises a specificity for an immune checkpoint molecule such as PD1. In at least one embodiment, the pharmaceutical composition comprises an anti-CD8 antibody and an additional active agent, wherein the additional active agent is an antibody comprising a specificity for an immune checkpoint molecule such as PD1, CTLA4, and LAG3.

In at least one embodiment, the pharmaceutical composition comprising an anti-CD8 antibody and an additional active agent, wherein the additional active agent is an antibody comprising a specificity for the immune checkpoint molecule PD1. Exemplary antibodies comprising a specificity for PD1 that are useful in the pharmaceutical composition embodiments disclosed herein include, but are not limited to, dostarlimab, pembrolizumab, nivolumab, and pidilizumab.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

In some embodiments, the formulation can be a sustained-release preparation of the antibody and/or other active ingredients. Suitable examples of sustained-release preparations include semipermeable matrices of solid hydrophobic polymers containing the antibody, which matrices are in the form of shaped articles, e.g. films, or microcapsules.

Typically, the formulations of the present disclosure to be administered to a subject are sterile. Sterile formulations may be readily prepared using well-known techniques, e.g., by filtration through sterile filtration membranes.

### IV. Uses and Methods of Treatment

It is contemplated that any of the compositions or formulations comprising an anti-CD8 antibody of the present disclosure can be used for any methods or uses, such as in therapeutic methods that utilize their ability to specifically bind to CD8, including CD8 expressed on CD8+ T cells, and thereby alter, mediate, and/or direct the function of CD8 as a cell surface receptor involved in immune regulation or signaling, particularly the function of CD8 in the immune response mediated by CD8+ T cells.

There are a range of diseases, disorders, and conditions that can potentially be treated by altering, mediating, and/or directing the immune regulatory and/or immune signaling activity of CD8 expressed on T cells. The range of diseases, disorders, and conditions include, but are not limited to, cancers, autoimmune disorders, and viral infections. For example, it is contemplated that any of the compositions or formulations comprising an anti-CD8 antibody of the present disclosure can be used for a method or use for the treatment of cancer, wherein the cancer is selected from colorectal cancer, pancreatic cancer, ovarian cancer, liver cancer, renal cancer, breast cancer, lung cancer, esophageal and gastric cancer, head and neck cancer, cervical cancer, prostate cancer, melanoma, bladder cancer, oral cancer, or hematological malignancies. In some embodiments, the present disclosure provides a method of treating cancer in a subject, the method comprising administering to the subject in need thereof a therapeutically effective amount of an anti-CD8 antibody of the present disclosure or administering to a subject a therapeutically effective amount of a pharmaceutical composition comprising an anti-CD8 antibody of the present disclosure and a pharmaceutically acceptable carrier.

As disclosed herein, including in the Examples below, the anti-CD8 antibodies of the present disclosure have the ability to specifically bind to CD8 expressed on CD8+ T cells, and thereby alter the therapeutic effect mediated by or afforded by CD8+ cells (including, but not limited to CD8+ T cells, NK cells, NKT cells). Accordingly, in some embodiments, the present disclosure provides a method of treating a CD8-mediated disease or condition in a subject, the method comprising administering to the subject a therapeutically effective amount of an anti-CD8 antibody of the present disclosure or administering to a subject in need thereof a therapeutically effective amount of a pharmaceutical composition comprising an anti-CD8 antibody of the present disclosure and a pharmaceutically acceptable carrier. Similarly, in some embodiments, the present disclosure provides a method of treating a disease mediated by binding to CD8 expressed on cells in a subject, the method comprising administering to the subject, the method comprising administering to the subject a therapeutically effective amount of an anti-CD8 antibody of the present disclosure or administering to a subject in need thereof a therapeutically effective amount of a pharmaceutical composition comprising an anti-CD8 antibody of the present disclosure and a pharmaceutically acceptable carrier.

A therapeutically effective amount of a pharmaceutical composition can comprise at least about 1 mg/kg of an anti-CD8 antibody or at least about 10 mg/kg of an anti-CD8 antibody. A therapeutically effective amount of a pharmaceutical composition can comprise at least about 2 mg/kg of an anti-CD8 antibody or at least about 20 mg/kg of an anti-CD8 antibody. A therapeutically effective amount of a pharmaceutical composition can comprise at least about 0.3 mg of an anti-CD8 antibody, at least about 1.0 mg of an anti-CD8 antibody, at least about 3.0 mg of an anti-CD8 antibody, at least about 10 mg of an anti-CD8 antibody, at least about 30 mg of an anti-CD8 antibody, at least about 100 mg of an anti-CD8 antibody, at least about 300 mg of an anti-CD8 antibody, at least about 900 mg of an anti-CD8 antibody, or at least about 1400 mg of an anti-CD8 antibody. A therapeutically effective amount of a pharmaceutical composition can comprise at least about 10 mg/kg of an anti-CD8 antibody, at least about 20 mg/kg of an anti-CD8 antibody, or at least about 100 mg/kg of an anti-CD8 antibody. In some embodiments, a therapeutically effective amount of a pharmaceutical composition can comprise an amount of an anti-CD8 antibody residing in a range between any two foregoing values.

In some embodiments, the therapeutically effective amount of a pharmaceutical composition can elicit no significant off-target effects. For example, in some cases there can be no significant off-target effects of an anti-CD8 antibody at a dose of not more than 10 mg/kg. As another example, in some cases there can be no significant off-target effects of an anti-CD8 antibody at a dose of not more than 20 mg/kg. or at a dose of not more than 100 mg/kg. In some embodiments, off target effects can include non-specific binding (e.g., as measured by BV ELISA) or cross-reactivity in human tissue. In some pharmaceutical compositions, there can be no significant off-target effects of an anti-CD8 antibody at another therapeutically effective amount provided herein, or within a range of any two provided therapeutically effective amounts provided herein.

Administration of the anti-CD8 antibody, composition, or pharmaceutical formulation in accordance with the method of treatment provides an antibody-induced therapeutic effect that protects the subject from and/or treats the progression of a CD8-mediated disease in a subject. In some embodiments, the method of treatment can further comprise administration of one or more additional therapeutic agents or treatments known to those of skill in the art to prevent and/or treat the CD8-mediated disease or condition. Such methods comprising administration of one or more additional agents can encompass combined administration (where two or more therapeutic agents are included in the same or separate formulations), and separate administration, in which case, administration of the antibody composition or formulation can occur prior to, simultaneously, and/or following, administration of the additional therapeutic agent.

In some embodiments of the methods of treatment of the present disclosure, the anti-CD8 antibody or pharmaceutical formulation comprising an anti-CD8 antibody is administered to a subject by any mode of administration that delivers the agent systemically, or to a desired target tissue. Systemic administration generally refers to any mode of administration of the antibody into a subject at a site other than directly into the desired target site, tissue, or organ, such that the antibody or formulation thereof enters the subject's circulatory system and, thus, is subject to metabolism and other like processes.

Accordingly, modes of administration useful in the methods of treatment of the present disclosure can include, but are not limited to, injection, infusion, instillation, and inhalation. Administration by injection can include intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal, intracerebro spinal, and intrasternal injection and infusion.

In some embodiments, a pharmaceutical formulation of the anti-CD8 antibody is formulated such that the antibody is protected from inactivation in the gut. Accordingly, the method of treatments can comprise oral administration of the formulation.

In some embodiments, use of the compositions or formulations comprising an anti-CD8 antibody of the present disclosure as a medicament are also provided. Additionally, in some embodiments, the present disclosure also provides for the use of a composition or a formulation comprising an anti-CD8 antibody in the manufacture or preparation of a medicament, particularly a medicament for treating, preventing or inhibiting a CD8-mediated disease. In a further embodiment, the medicament is for use in a method for treating, preventing or inhibiting a CD8-mediated disease comprising administering to an individual having a CD8-mediated disease an effective amount of the medicament. In certain embodiments, the medicament further comprises an effective amount of at least one additional therapeutic agent, or treatment. Exemplary additional therapeutic agents or treatments that can be used in such medicaments can include but are not limited to an antibody comprising a specificity for an immune checkpoint molecule such as PD1, CTLA4, and LAG3. In at least one embodiment, the additional therapeutic agent or treatment present in a medicament of the present disclosure is an antibody comprising a specificity for the immune checkpoint molecule PD1, including but not limited to an antibody selected from dostarlimab, pembrolizumab, nivolumab, and pidilizumab.

In a further embodiment, the medicament is for use in treating, inhibiting or preventing a CD8-mediated disease in a subject comprising administering to the subject an amount effective of the medicament to treat, inhibit or prevent the CD8-mediated disease.

For the prevention or treatment of a CD8-mediated disease or condition, the appropriate dosage of the anti-CD8 antibody contained in the compositions and formulations of the present disclosure (when used alone or in combination with one or more other additional therapeutic agents) will depend on the specific disease or condition being treated, the severity and course of the disease, whether the antibody is administered for preventive or therapeutic purposes, the previous therapy administered to the patient, the patient's clinical history and response to the antibody, and the discretion of the attending physician. The anti-CD8 antibody included in the compositions and formulations described herein, can be suitably administered to the patient at one time, or over a series of treatments. Various dosing schedules including but not limited to single or multiple administrations over various time-points, bolus administration, and pulse infusion are contemplated herein.

Depending on the type and severity of the disease, about 1 μg/kg to 20 mg/kg of anti-CD8 antibody in a formulation of the present disclosure is an initial candidate dosage for administration to a human subject, whether, for example, by one or more separate administrations, or by continuous infusion. Generally, the administered dosage of the antibody can be in the range from about 0.05 mg/kg to about 20 mg/kg. In some embodiments, one or more doses of about 0.5 mg/kg, 2.0 mg/kg, 4.0 mg/kg 10 mg/kg, 20 mg/kg, or a range between any two foregoing values (or any combination thereof) may be administered to a human subject. In some embodiments, a dose administered to a human subject can be greater than about 20 mg/kg.

In some embodiments, a therapeutically effective amount can be administered to a subject, such as a human subject. A therapeutically effective amount can be at least about 1 mg/kg, at least about 10 mg/kg, at least about a dose between about 1 mg/kg and about 10 mg/kg. A therapeutically effective amount can be at least about 2 mg/kg, at least about 20 mg/kg, or at least about a dose between about 2 mg/kg and about 20 mg/kg. A therapeutically effective amount can be at least about 0.3 mg, at least about 1.0 mg, at least about 3.0 mg, at least about 10 mg, at least about 30 mg, at least about 100 mg, at least about 300 mg, at least about 900 mg, at least about 1400 mg/kg, or at least about a dose residing in a range between any two foregoing values. A therapeutically effective amount can be at least about 10 mg/kg, at least about 20 mg/kg, at least about 100 mg/kg, or at least about a dose residing in a range between any two foregoing values.

Dosage administration can be maintained over several days or longer, depending on the condition of the subject, for example, administration can continue until the CD8-mediated disease is sufficiently treated, as determined by methods known in the art. In some embodiments, an initial higher loading dose may be administered, followed by one or more lower doses (e.g., one or more maintenance doses). However, other dosage regimens may be useful. The progress of the therapeutic effect of dosage administration can be monitored by conventional techniques and assays.

Accordingly, in some embodiments of the methods of the present disclosure, the administration of the anti-CD8 antibody comprises a daily dosage from about 1 mg/kg to about 100 mg/kg. In some embodiments, the dosage of anti-CD8 antibody comprises a daily dosage of at least about 1 mg/kg, at least about 5 mg/kg, at least about 10 mg/kg, at least about 20 mg/kg, or at least about 30 mg/kg.

### EXAMPLES

Various features and embodiments of the disclosure are illustrated in the following representative examples, which are intended to be illustrative, and not limiting. Those skilled in the art will readily appreciate that the specific examples are only illustrative of the invention as described more fully in the claims which follow thereafter. Every embodiment and feature described in the application should be understood to be interchangeable and combinable with every embodiment contained within.

### Example 1: Cloning and Expression of Human CD8A Antigen

To generate an antibody targeting the extracellular domain (ECD) of human CD8, gene fragments encoding amino acids 22-182 of the T-cell surface CD8 alpha chain (“hu-CD8A, Uniprot ID: P01732) identified herein as SEQ ID NO: 1, were reverse-transcribed in silico and then synthesized and cloned into mammalian expression vector with (His)x6 tag or Fc tag at the C-terminus with standard molecular biology techniques.

Hu-CD8A antigen with (His)x6 or Fc fusions were expressed in Chinese hamster ovary (CHO) cells. ExpiCHO cells (Thermo Fisher Scientific) were maintained at 37° C. with 5% CO₂ and 130 rpm in 60 mL of ExpiCHO expression medium in Erlenmeyer flasks. Transfection was performed according to instructions provided with the ExpiCHO™ Expression System Kit (ThermoFisher, Catalog number A29133). Briefly, when cells reached Viable Cell Density (VCD) of 6×10⁶ cells/mL and doubling time of 18-20 h, 1 mg plasmid DNA per 10⁶ cells and 0.8 mg/ml ExpiFectamine CHO was mixed by repeated inversions, diluted with cold OptiPRO serum free medium, and finally complexed to the diluted plasmid DNA at room temperature. After 5 min, the mix was added drop by drop to the cell culture at room temperature. After transfection, cells were cultured at 130 RPM for 25 ml or large flasks. After 7-12 days post transfection, culture media were centrifuged at 25° C. and 500×g for 5 min to pellet cells and then at 4° C. and 4500×g for 30 min. The clarified supernatant was filtered by 0.45 um filters and fusion proteins were purified with Ni⁺⁺ or protein A affinity column chromatography (Example 2).

### Example 2: Purification of Human CD8A ECD Antigen

Hu-CD8A ECD polypeptide (SEQ ID NO: 1) with His-tag were purified by affinity chromatography with nickel resin (Ni Sepharose excel, Cytiva). Dripping columns packed with nickel resin (column volume (CV)=2 mL) were equilibrated with 5× CV of PBS buffer and 100 mL supernatant was directly loaded. Unbound protein was removed by washing with 10 CV of PBS buffer and then 10 CV of PBS buffer containing 20 mM Imidazole. The protein of interest was eluted with 5 CV of PBS buffer with 500 mM Imidazole.

The hu-CD8A antigen was further polished with cation-exchange chromatography (CEX) purification step on an ÄKTA Avant 25 system (Cytiva). The His-tag purified CD8 antigen was diluted 10× with 20 mM sodium phosphate buffer (pH 6.3) and loaded on CEX column packed with SP-FF resin (column volume=5 mL). After 5-column volume washing with buffer A, the target protein was eluted with 20 CV buffer B (20 mM sodium phosphate+1M NaCl, pH6.3) with a linear gradient from 0 to 50% or 10-50%.

### Example 3: Immunization and Generation of Anti-CD8 VHH Sequences

Antigen of recombinant hu-CD8A ECD polypeptide (SEQ ID NO: 1) was expressed in CHO cells with the following C-terminal Fc tag sequence of SEQ ID NO: 106:

(SEQ ID NO: 106)
EPKTPKPQPQPQPQPNPTTESKCPKCPAPELLGGPSVFIFPPKPK
DVLSISGRPEVTCVVVDVGQEDPEVSFNWYIDGAEVRTANTRPKE
EQFNSTYRVVSVLPIQHQDWLTGKEFKCKVNNKALPAPIEKTISK
AKGQTREPQVYALAPHREELAKDTVSVTCLVKGFYPPDINVEWOR
NROPEPEGTYATTPPQLDNDGTYFLYSKLSVGKNTWORGETFTCV
VMHETLHNHYTQKSISQS.

Immunization of llamas with hu-CD8A was performed using standard protocol, with three boosts after the initial immunization with 1 mg of antigen per llama. The titer of serum antibodies was measured by ELISA assays. A high anti-llama IgG2/3 serum titer (1:2,000) using ELISA assay was observed after immunization. Whole blood was then collected, and PBMCs were isolated. mRNA was isolated from the PBMCs using PureLink RNA mini kit (Invitrogen).

The VHH genes were amplified by PCR using standard llama IgG primers. PCR products were purified by QIAquick gel extraction kit (Qiagen), constructed into a phagemid vector pADL-23c (Antibody Design Laboratories) and transformed to TG1 electrocompetent cells (Lucigen). Phages with VHH displayed on surface were produced by adding M13 helper phage. Three rounds of panning were performed using streptavidin coupled Dynabeads (Thermo Fisher Scientific) coated with biotinylated human CD8 alpha. Eluted phage from last rounds of panning were used to infect TG1 cells. Single colonies of TG1 cells were picked and cultured in 2xYT medium, and IPTG was added for secretion of VHH antibodies. Supernatants with VHH antibodies were screened by ELISA assays using hu-CD8A ECD coated plates. Positive hu-CD8A binders as determined by ELISA were picked for sequencing. A total 10 unique anti-CD8 VHH sequences were identified and grouped into 4 groups using Uniprot Sequence alignment tool. The VH domain, CDR-H1, CDR-H2, and CDR-H3 sequences of these positive binding hits are shown in Table 3 and the accompanying Sequence Listing.

### Example 4: Cloning and Expression of Anti-CD8 VHH Antibodies

Positive anti-CD8 VHH sequences identified from the clones of Example 3 were formatted as bivalent VHH antibodies by fusing the VHH to the N-terminus of the hinge and Fc region of human IgG1, including human constant heavy chain 2 (CH2) and constant heavy chain 3 (CH3) domains of SEQ ID NO: 107 (Uniprot ID: P01857).

(SEQ ID NO: 107)
DKTHTCPPCPAPELLGGPSVFLFPPKPKDTLMISRTPEVTCVVVD
VSHEDPEVKENWYVDGVEVHNAKTKPREEQYNSTYRVVSVLTVLH
QDWLNGKEYKCKVSNKALPAPIEKTISKAKGQPREPQVYTLPPSR
DELTKNQVSLTCLVKGFYPSDIAVEWESNGQPENNYKTTPPVLDS
DGSFFLYSKLTVDKSRWQQGNVFSCSVMHEALHNHYTQKSLSLSP
GK

The resulting bivalent anti-CD8 VHH antibody constructs were cloned in pcDNA3.1 (+) vector and expressed in Chinese hamster ovary (CHO) cells using ExpiCHO expression system (Thermo Fisher Scientific). ExpiCHO cells (Thermo Fisher Scientific) were maintained at 37° C., 5% CO₂ and 130 rpm in 60 mL of ExpiCHO expression medium in Erlenmeyer flasks. Transfection was performed according to instructions provided with the ExpiCHO™ Expression System Kit (ThermoFisher—Catalog number A29133). Briefly, when cells reached Viable Cell Density (VCD) of 6×10⁶ cells/mL and doubling time of 18-20 h, 1 μg plasmid DNA per 10⁶ cells and ExpiFectamine CHO was mixed by repeated inversions, diluted with cold OptiPRO serum free medium, and finally complexed to the diluted plasmid DNA at room temperature. After 5 min, the mix was added drop by drop to the cell culture at room temperature. After transfection, cells were cultured at 130 RPM for 25 mL or large flasks. After 7-12 days post transfection, culture media were centrifuged at 25° C. and 500×g for 5 min to pellet cells and then at 4° C. and 4500×g for 30 min. The clarified supernatant was filtered by 0.45 μm filtration and the VHH antibodies were purified with protein A affinity column chromatography.

### Example 5: Purification of Anti-CD8 VHH Antibodies

The CHO cell-expressed bivalent anti-CD8 VHH antibody constructs were purified by affinity chromatography with protein-A (Atmosphere A3, JSR Life Sciences). Dripping columns packed with protein A resin (column volume=0.2 mL) were equilibrated with 5 CV 1×PBS buffer and 4 mL supernatant was directly loaded. Unbound protein was removed by washing with 10 column volumes 1× PBS buffer, and the protein of interest was eluted. The eluted fractions were collected, and pH was adjusted using 1M Tris buffer, pH 8.0.

### Example 6: SEC-HPLC Characterization of Anti-CD8 VHH Antibodies

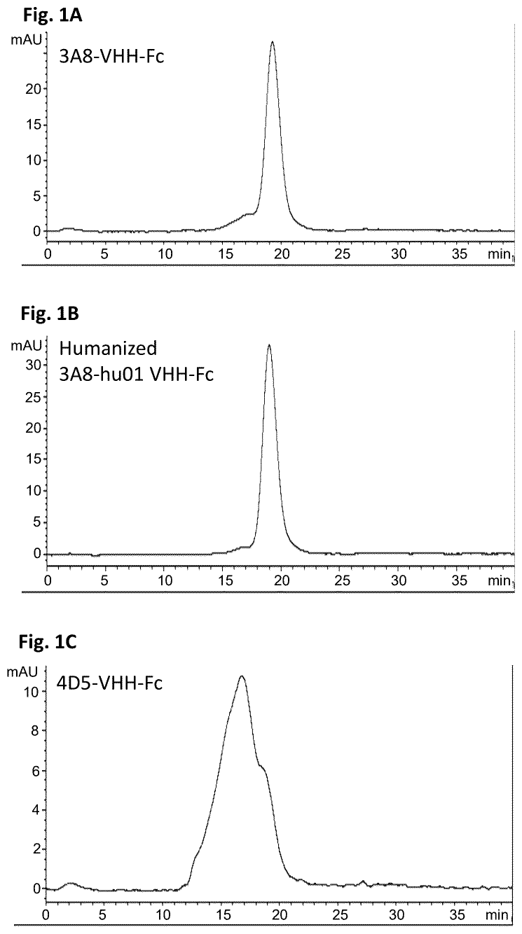

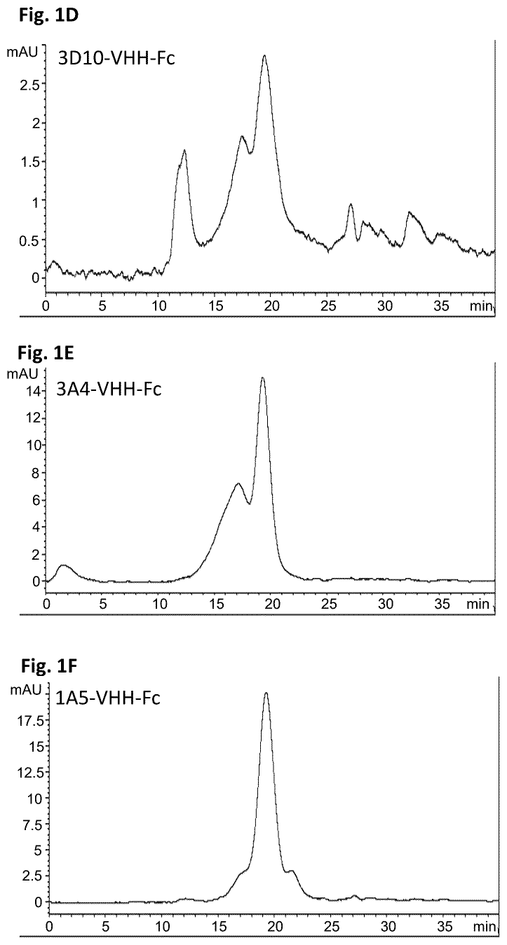

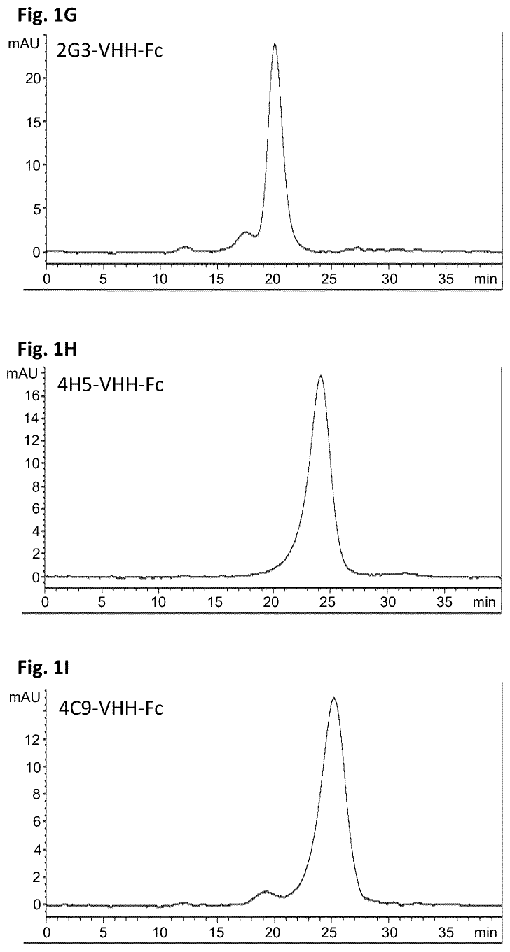

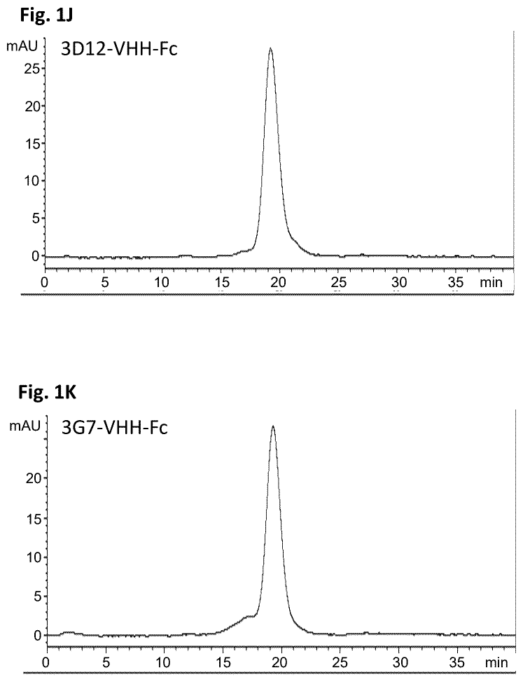

Size exclusion-high-performance liquid chromatography (SEC-HPLC) analysis was performed on an Agilent 1100 HPLC system (Santa Clara, California) with 1×PBS as a running buffer. Purified VHH antibody samples were injected into a prepacked Superose 12-300 column (Cytiva) which was equilibrated with 1×PBS buffer. The flow rate was 0.65 mL/min and the total running time was 40 min. The SEC-HPLC elution profiles for the various anti-CD8 VHH antibodies are shown in FIGS. 1A, 1C, 1D, 1E, 1F, 1G, 1H, 1I, 1J, and 1K. Additionally, the SEC-HPLC profile of the humanized version of anti-CD8 VHH antibody 3A8 (hu01 prepared as described below) is shown in FIG. 1B.

### Example 7: CD8A ECD Affinity Assessment of Anti-CD8 VHH Antibodies

Biolayer interferometry was used to measure the association and dissociation constants for the binding kinetics of the bivalent anti-human CD8A VHH antibodies. The binding kinetics was measured on a Gator Prime system (Gator Bio, Inc.) at 30° C. and analyzed with the Gator Prime software. Anti-human-IgG Fc (HFC) probe (Gator Bio, Inc.) was used to capture the anti-hu-CD8 VHH antibodies or control antibodies. A kinetic buffer only well was set as a reference well for subtraction during data processing. Data were fitted with a 1:1 Langmuir model for association and dissociation using Rmax linked global fitting for each antibody-antigen binding. Human CD8A/B with His tag is recombinant CD8 alpha and beta heterodimer protein with a poly histidine tag fused to C-terminus (Sino Biological US Inc.). A total of 10 anti-CD8 VHH antibodies were found to effectively bind the human CD8A/B ECD heterodimer with His tag. Their affinity is shown in Table 4 below.

TABLE 4
Anti-CD8 VHH antibody binding affinity
for hu-CD8A/B heterodimer

| VHH Groups | VHH mAb clone | Kd (M) | kon(1/Ms) | Koff (1/s) |
|---|---|---|---|---|
| Group 1 | 3A8 | 8.84E−09 | 3.01E+05 | 0.00266 |
| | 3A8-hu01 | 8.70E−09 | 3.40E+05 | 0.00296 |
| | 4D5 | 6.08E−09 | 3.18E+05 | 0.00193 |
| | 3D10 | 8.25E−09 | 2.74E+05 | 0.00226 |
| | 3A4 | 8.59E−09 | 2.74E+05 | 0.00236 |
| Group 2 | 1A5 | 1.18E−08 | 2.40E+05 | 0.00282 |
| | 2G3 | 4.45E−08 | 2.14E+05 | 0.00953 |
| Group 3 | 4H5 | 4.36E−08 | 1.80E+05 | 0.00786 |
| | 4C9 | 3.47E−09 | 3.34E+05 | 0.00116 |
| Group 4 | 3D12 | 8.38E−09 | 3.73E+05 | 0.00313 |
| | 3G7 | 2.38E−08 | 2.13E+05 | 0.00507 |

Antigen used in binding: ECD of human CD8A/B heterodimer with his tag

### Example 8: Affinity of Anti-CD8 VHH Antibodies for Cynomolgus CD8A ECD

To understand the species cross-reactivity to cyno-CD8A, the binding kinetics of anti-CD8 VHH-Fc antibodies to cyno-CD8A were also measured on Gator Prime. A recombinant cyno-CD8A ECD polypeptide of SEQ ID NO: 108 (UniProt A0A2K5ULI8) fused at its C-terminus to a human Fc tag was used as antigen (Sino Biological US Inc.).

(SEQ ID NO: 108)
NQFRVSPLGRTWNLGETVELKCQVLLSNPTSGCSWLFQPRGTAAR
PTFLLYLSQNKPKAAEGLDTQRFSGKRLGDTFVLTLRDFRQENEG
YYFCSALSNSIMYFSHFVPVFPCSEAHHYASAATTHTGAHHRVAA
PVPAPRGVPASGGGSVNTRGLDFACD

As shown in Table 5 (below), all of the anti-CD8 VHH clones were found to be cross-reactive with the cyno-CD8A ECD antigen.

TABLE 5
Binding affinity of anti-CD8 VHH antibodies
to the ECD of cyno CD8A.

| VHH groups | VHH mAb clone | Kd (M) | kon(1/Ms) | Koff (1/s) |
|---|---|---|---|---|
| Group 1 | 3A8 | 9.21E−11 | 1.11E+06 | 0.000102 |
| | 3A8-hu01 | 5.98E−10 | 2.07E+05 | 0.000124 |
| | 4D5 | 1.43E−10 | 8.98E+05 | 0.000128 |
| | 3D10 | 7.95E−10 | 5.44E+05 | 0.000432 |
| | 3A4 | 5.68E−10 | 5.30E+05 | 0.000301 |
| Group 2 | 1A5 | 1.18E−08 | 2.40E+05 | 0.00282 |
| | 2G3 | 4.45E−08 | 2.14E+05 | 0.00953 |
| Group 3 | 4H5 | 4.36E−08 | 1.80E+05 | 0.00786 |
| | 4C9 | 9.65E−11 | 1.05E+06 | 0.000102 |
| Group 4 | 3D12 | 5.47E−11 | 2.32E+06 | 0.000127 |
| | 3G7 | 9.37E−10 | 4.61E+05 | 0.000432 |

Antigen used in binding: ECD of cynomolgus monkey CD8A with Fc tag

### Example 9: Generation of a Stable CD8+ Cell Line

HEK Blue cells (Invivogen, Inc., catalog number: hkb-il2) were infected at MOI 20 with hu-CD8A (NM_001768) ORF Clone Lentiviral Particle (Origene Catalog number: RC206608L3V). About 60% CD8A positive (measured by flow with BV421-CD8 antibody) transduced cells went through 2 rounds of single cell cloning (limiting dilutions at 0.3 cells/well), with 150 ml of DMEM medium with 10% FBS in 96-well plate), cultured at 37° C. with 5% CO2. Cell clones above 95% positive for CD8 were picked for amplifying and used in the later cell-based assay.

### Example 10: CD8+ HEK Blue Cell Binding Assay of Anti-CD8 VHH Antibodies

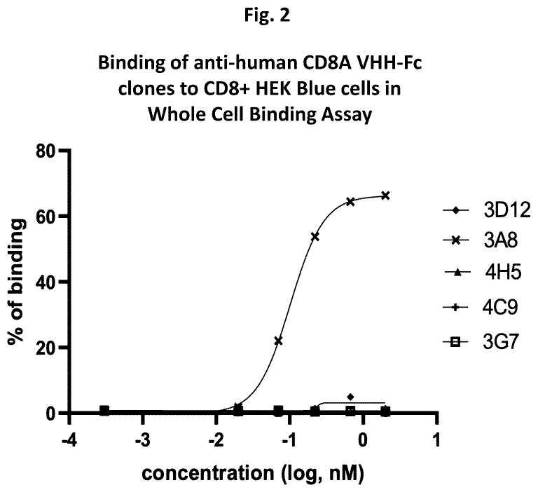

Cell suspension with above 90% viability were prepared for HEKBlue-CD8+. Testing molecules at concentrations indicated were serially diluted in FACS buffer (PBS w 2% FBS) in 40 μL/well flat-bottom 96-well plate. 50K relevant cell suspensions in 40 μL were added to each well, incubated at 37° C. for 30 min. The cells were spun down and stained with 1:500 FITC or APC conjugated goat anti-human Fc for another 30 min at 4° C., washed once with FACS buffer and FACS analysis were performed with CytoFlex (Beckman Coulter, Inc). As shown in FIG. 2, only the anti-CD8 VHH antibody of clone 3A8 was found to bind human CD8A when expressed on the cell surface with an EC₅₀ at 0.101 nM.

### Example 11: Humanization of Anti-CD8 VHH Antibodies

To humanize the anti-CD8 VHH antibody clone 3A8, the Igblast tool from NCBI was used to blast the AA sequence of this clone against the NCBI database of human germline VH genes. Closest human germline sequences were selected, and mutations were made to change the framework sequences from llama to human while keeping the original CDRs. For the antibody of the 3A8 clone, the human germline VH gene IGHV3-23*01 was found to have the highest homology and hence was used. The frequency of individual amino acids in the 3A8 framework regions were also checked using AbYsis human immunoglobin database shown in Table 6 (below). The AA residues with very low frequency in human VH genes (<1% of sequences) were replaced by one or more of the prevalent residues in human VH genes, based on frequencies shown in Table 6 (below).

TABLE 6
AA Frequency in Llama Anti-CD8 VHH
3A8 and Top AA in Human VH Domain

| AA in in | | AA frequency | | 2^{nd} |
|---|---|---|---|---|
| human VH | Framework | of 3A8 in | Top AA in | AA in |
| (Kabat #) | (FR) | human VH | human VH | human VH |
| H01 | FR1 | Q (50%) | E (39%) | |
| H05 | | Q (34%) | V (38%) | L (11%) |
| H14 | | A (2%) | P (95%) | |
| H37 | FR2 | F (2%) | V (75%) | I (20%) |
| H44 | | E (1%) | G (86%) | |
| H45 | | R (2%) | L (96%) | |
| H47 | | G (<1%) | W (94%) | |
| H68 | FR3 | A (1%) | T (85%) | |
| H71 | | S (1%) | R (48%) | V (21%) |
| H74 | | A (14%) | S (70%) | |
| H78 | | V (16%) | A (34%) | L (32%) |
| H82b | | N (7%) | S (83%) | |
| H83 | | K (12%) | T (41%) | R (39%) |
| H84 | | P (9%) | A (38%) | S (34%) |
| H108 | FR4 | Q (3%) | L (48%) | T (28%) |

The amino acid sequences of 28 exemplary humanized versions of the clone 3A8 anti-CD8 VHH antibody (denoted as clones “3A8-hu01” through “3A8-hu28”) are provided in Table 3 and the accompanying Sequence Listing.

One example of these humanized versions of the anti-CD8 VHH antibody, 3A8 is clone 3A8-hu01 (SEQ ID NO: 78). The humanized anti-CD8 VHH 3A8-hu01 of SEQ ID NO: 78 has the following framework amino acid substitutions relative to the anti-CD8 VHH parent antibody, 3A8 of SEQ ID NO: 2 (Kabat numbering system): Q1E, Q5V, A14P, A68T, S71R, A74S, N82bS, K83R, P84A. A gene encoding this humanized 3A8-hu01 of SEQ ID NO: 78 was cloned into pcDNA3.1 (+) expression vector (Thermo Fisher Scientific), and the humanized anti-CD8 VHH antibody protein was produced by transient transfection of ExpiCHO, then purified by protein A affinity chromatography as described in the Examples above. The 3A8-hu01 was analyzed by SEC-HPLC (as described in example above) and its measured profile is shown in FIG. 1B. A comparison of its SEC-HPLC profile with its parent antibody's profile (FIG. 1A) shows that 3A8-hu01 has a similar or higher percentage of monomer. The binding affinities of the 3A8-hu01 to the hu-CD8A/B and cyno-CD8A antigens were also determined (as described in the Examples above) and are listed above in Tables 4 and 5, respectively. No significant change in binding affinity was observed between the humanized 3A8-hu01 and the llama antibody parent 3A8.

While the foregoing disclosure of the present invention has been described in some detail by way of example and illustration for purposes of clarity and understanding, this disclosure including the examples, descriptions, and embodiments described herein are for illustrative purposes, are intended to be exemplary, and should not be construed as limiting the present disclosure. It will be clear to one skilled in the art that various modifications or changes to the examples, descriptions, and embodiments described herein can be made and are to be included within the spirit and purview of this disclosure and the appended claims. Further, one of skill in the art will recognize a number of equivalent methods and procedure to those described herein. All such equivalents are to be understood to be within the scope of the present disclosure and are covered by the appended claims.

Additional embodiments of the invention are set forth in the following claims.

The disclosures of all publications, patent applications, patents, or other documents mentioned herein are expressly incorporated by reference in their entirety for all purposes to the same extent as if each such individual publication, patent, patent application or other document were individually specifically indicated to be incorporated by reference herein in its entirety for all purposes and were set forth in its entirety herein. In case of conflict, the present specification, including specified terms, will control.

What is claimed is:

1. An anti-CD8 antibody comprising a single heavy chain variable domain (VH) comprising a first heavy chain complementarity region (CDR-H1), a second heavy chain complementarity region (CDR-H2), and a third heavy chain complementarity region (CDR-H3), wherein CDR-H1, CDR-H2, and CDR-H3, respectively, have amino acid sequences of:
(a) SEQ ID NO: 3, 4, and 5;
(b) SEQ ID NO: 7, 8, and 9;
(c) SEQ ID NO: 11, 12, and 13;
(d) SEQ ID NO: 15, 16, and 17;
(e) SEQ ID NO: 19, 20, and 21;
(f) SEQ ID NO: 23, 24, and 25;
(g) SEQ ID NO: 27, 28, and 29;
(h) SEQ ID NO: 31, 32, and 33;
(i) SEQ ID NO: 35, 36, and 37;
(j) SEQ ID NO: 39, 40, and 41;
(k) SEQ ID NO: 43, 44, and 45;
(l) SEQ ID NO: 47, 48, and 49;
(m) SEQ ID NO: 51, 52, and 53;
(n) SEQ ID NO: 55, 56, and 57;
(o) SEQ ID NO: 59, 60, and 61;
(p) SEQ ID NO: 63, 64, and 65;
(q) SEQ ID NO: 67, 68, and 69;
(r) SEQ ID NO: 71, 72, and 73; or
(s) SEQ ID NO: 75, 76, and 77.

2. The antibody of claim 1, wherein the antibody comprises a heavy chain variable domain (VH) amino acid sequence having at least 90% identity to a sequence selected from SEQ ID NO: 2, 6, 10, 14, 18, 22, 26, 30, 34, 38, 42, 46, 50, 54, 58, 62, 66, 70, and 74.

3. The antibody of claim 1, wherein the antibody is humanized.

4. The antibody of claim 3, wherein the antibody comprises a heavy chain variable domain (VH) amino acid sequence having at least 90% identity to a sequence selected from SEQ ID NO: 78, 78, 79, 80, 81, 82, 83, 84, 85, 86, 87, 88, 89, 90, 91, 92, 93, 94, 95, 96, 97, 98, 99, 100, 101, 102, 103, 104, and 105.

5. The antibody of claim 1, wherein the antibody is an antibody fragment; optionally, wherein the antibody fragment is selected from: a VHH antibody, a scFv antibody, a single-domain antibody, a Fv fragment, a Fab fragment, Fab′ fragment, Fab′-SH fragment, a F(ab′)₂ fragment, a diabody, a linear antibody, a single-armed antibody, and a single-chain antibody.

6. The antibody of claim 1, wherein the antibody comprises a fusion; optionally, wherein the fusion comprises a portion or all of an immunoglobulin Fc region, a cytokine protein, and/or another antibody.

7. The antibody of claim 6, wherein the fusion comprises a polypeptide linker; optionally, wherein the polypeptide linker comprises an amino acid sequence selected from (GGGGS)ₙ, (SSSSG)ₙ, (GGGG)(SGGGG)ₙ, (EAAAK)ₙ, and (XP)ₙ, ENLYFQ(−G/S), where n is 1 to 10.

8. The antibody of claim 1, wherein the antibody is characterized by one or more of the following properties:
(a) binds to hu-CD8 with a binding affinity of 1×10⁻⁸ M or less, 1×10⁻⁹ M or less, 1×10⁻¹⁰ M or less, or 1×10⁻¹¹ M or less; optionally, wherein the binding affinity is measured by equilibrium dissociation constant (K_{D}) to a hu-CD8 polypeptide of SEQ ID NO: 1;
(b) binds to cyno-CD8 with a binding affinity of 1×10⁻⁸ M or less, 1×10⁻⁹ M or less, 1×10⁻¹⁰ M or less, or 1×10⁻¹¹ M or less; optionally, wherein the binding affinity is measured by equilibrium dissociation constant (K_{D}) to a cyno-CD8 polypeptide of SEQ ID NO: 108; and/or
(c) binds to CD8+ cells with an EC₅₀ of 2.5 nM or less, 1 nM or less, 0.5 nM or less, or 0.25 nM or less.

9. An anti-CD8 antibody that specifically binds to the same epitope as the antibody of claim 1; optionally, wherein the antibody binds to the same epitope as the antibody of clone 3A8.

10. An isolated polynucleotide or vector encoding the antibody of any one of claim 1.

11. An isolated host cell comprising the polynucleotide or vector of claim 10; optionally, wherein the host cell is selected from a Chinese hamster ovary (CHO) cell, a myeloma cell (e.g., Y0, NS0, Sp2/0), a monkey kidney cell (COS-7), a human embryonic kidney line (293), a baby hamster kidney cell (BHK), a mouse Sertoli cell (e.g., TM4), an African green monkey kidney cell (VERO-76), a human cervical carcinoma cell (HELA), a canine kidney cell, a human lung cell (W138), a human liver cell (Hep G2), a mouse mammary tumor cell, a TR1 cell, a Medical Research Council 5 (MRC 5) cell, and a FS4 cell.

12. A method of producing an antibody comprising culturing the host cell of claim 11 so that an antibody is produced.

13. A pharmaceutical composition comprising an anti-CD8 antibody of claim 1 and a pharmaceutically acceptable carrier.

14. The composition of claim 13, wherein the composition further comprises a chemotherapeutic agent or another antibody comprising a specificity for an immune checkpoint molecule.

15. A method of treating a CD8 mediated disease in a subject, the method comprising administering to the subject a therapeutically effective amount of an antibody of claim 1, or administering to the subject a therapeutically effective amount of a pharmaceutical composition of claim 13.

16. The method of claim 15, wherein the therapeutically effective amount is at least about 1 mg/kg, at least about 2 mg/kg, at least about 10 mg/kg, at least about 20 mg/kg, or at least about 100 mg/kg.

17. The method of claim 15, wherein the disease is a cancer; optionally, wherein the cancer is selected from colorectal cancer, pancreatic cancer, ovarian cancer, liver cancer, renal cancer, breast cancer, lung cancer, esophageal and gastric cancer, head and neck cancer, cervical cancer, prostate cancer, melanoma, bladder cancer, oral cancer, or hematological malignancies.

18. The method of claim 15, wherein the disease is an autoimmune disease; optionally, wherein the autoimmune disease is selected from Crohn's disease, ulcerative colitis, celiac disease, systemic lupus erythematosus, psoriatic arthritis, rheumatoid arthritis, Sjogren's syndrome, type 1 diabetes, atopic dermatitis, psoriasis, and multiple sclerosis.


SEQUENCE LISTING (108 sequences)

SEQ ID NO: 1 - protein, 161 aa, Homo sapiens
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACD

SEQ ID NO: 2 - protein, 122 aa, synthetic construct
QVQLQESGGGLVQAGGSLRLSCAASGFTFDEYAIGWFRQAPGKEREGVSCLRVSDGRTYYPDSVKGRFAISSDNAKNTVYLQMNNLKPEDTAVYYCASGSYYGCTVDDYDFWGQGTQVTVSS

SEQ ID NO: 3 - protein, 5 aa, Synthetic construct
EYAIG

SEQ ID NO: 4 - protein, 17 aa, Synthetic construct
CLRVSDGRTYYPDSVKG

SEQ ID NO: 5 - protein, 13 aa, synthetic construct
GSYYGCTVDDYDF

SEQ ID NO: 6 - protein, 122 aa, synthetic construct
QVQLQESGGGLVQAGGSLRLSCAASGFTFDEYAIGWFRQAPGKEREGVSCLRVSDGRTYYPDSVKGRFAISSDNAKNTVYLQMNNLKPEDTAVYYCASGSYYGCTVHDYDFWGQGTLVTVSS

SEQ ID NO: 7 - protein, 5 aa, Synthetic construct
EYAIG

SEQ ID NO: 8 - protein, 17 aa, synthetic construct
CLRVSDGRTYYPDSVKG

SEQ ID NO: 9 - protein, 13 aa, Synthetic construct
GSYYGCTVHDYDF

SEQ ID NO: 10 - protein, 122 aa, Synthetic construct
QVQLQESGGGLVQAGGSLRLSCAASGFTFDEYAIGWFRQAPGKEREGVSCLRVSDGRTYYPDSVKGRFAISSDNAKNTVYLQMNNLKPEDTAVYYCASGSYYGCTVHDYDFWGQGTQVTVSS

SEQ ID NO: 11 - protein, 5 aa, Synthetic construct
EYAIG

SEQ ID NO: 12 - protein, 17 aa, Synthetic construct
CLRVSDGRTYYPDSVKG

SEQ ID NO: 13 - protein, 13 aa, Synthetic construct
GSYYGCTVHDYDF

SEQ ID NO: 14 - protein, 122 aa, Synthetic construct
QVQLQESGGGLVQAGGSLRLSCAASGFTFNEYAIGWFRQAPGKEREGVSCLRVSDGRTYYPYSVKGRFAISNDNANNTVYLQMNNLKPEDTAVYYCASGSYYGCTVDDYDFWGQGTQVTVSS

SEQ ID NO: 15 - protein, 5 aa, Synthetic construct
EYAIG

SEQ ID NO: 16 - protein, 17 aa, Synthetic construct
CLRVSDGRTYYPYSVKG

SEQ ID NO: 17 - protein, 13 aa, Synthetic construct
GSYYGCTVDDYDF

SEQ ID NO: 18 - protein, 124 aa, Synthetic construct
QVQLQESGGGLVQAGGSLKLSCAASGFTFNDYAIGWFPQAPGKEREGVSCIINIDGSTYYADSVKGRFPISSDNAKNTVYLQMNSLKPEDTAVYYCAAEGLHSCRDLYYGMDYWGKGTLVTVSS

SEQ ID NO: 19 - protein, 5 aa, Synthetic construct
AIGWF

SEQ ID NO: 20 - protein, 17 aa, Synthetic construct
CIINIDGSTYYADSVKG

SEQ ID NO: 21 - protein, 15 aa, Synthetic construct
EGLHSCRDLYYGMDY

SEQ ID NO: 22 - protein, 124 aa, Synthetic construct
QVQLQESGGGLVQPGGSLKLSCAASGFTFYDYAIGWFRQAPGKEREGVSCISNIDGSTYYADSVKGRFTISTDNAKNTVYLQMNSLKPEDTAVYYCAAEGLHSCRDLYYGMDYWGKGTLVTVSS

SEQ ID NO: 23 - protein, 5 aa, Synthetic construct
AIGWF

SEQ ID NO: 24 - protein, 17 aa, Synthetic construct
CISNIDGSTYYADSVKG

SEQ ID NO: 25 - protein, 15 aa, Synthetic construct
EGLHSCRDLYYGMDY

SEQ ID NO: 26 - protein, 124 aa, Synthetic construct
QVQLQESGGGLVQAGGSLRLSCAASGFTFDDYAIGWFRQAPGKEREGVSCISSIDGSTYYADSVKGRFTISRDNDKNTVYLQMNSLKPEDTAVYYCTAEGLHSCRDLYYGMDYWGKGTQVTVSS

SEQ ID NO: 27 - protein, 5 aa, Synthetic construct
AIGWF

SEQ ID NO: 28 - protein, 17 aa, Synthetic construct
CISSIDGSTYYADSVKG

SEQ ID NO: 29 - protein, 15 aa, Synthetic construct
EGLHSCRDLYYGMDY

SEQ ID NO: 30 - protein, 124 aa, Synthetic construct
QVQLQESGGGLVQAGGSLRLSCAASGVPFDDYAIGWFRQAPGKEREGVSCISNIDGSTYYADSVKGRFTISSDNAKNTVYLQMNSLKPEDTAVYYCAAEGLHSCRDLYYGMDYWGKGTLVTVSS

SEQ ID NO: 31 - protein, 5 aa, Synthetic construct
AIGWF

SEQ ID NO: 32 - protein, 17 aa, Synthetic construct
CISNIDGSTYYADSVKG

SEQ ID NO: 33 - protein, 15 aa, Synthetic construct
EGLHSCRDLYYGMDY

SEQ ID NO: 34 - protein, 124 aa, Synthetic construct
QVQLQESGGGLVQAGGSLRLSCAASGFTFDDYAIGWFRQAPGKEREGVSCISNIDGHTYYADSVKGRFTISSDNAKNTVYLQMNSLKPEDTAVYYCAAEGLHSCKDLYYGMDYWGKGTLVTVSS

SEQ ID NO: 35 - protein, 5 aa, Synthetic construct
AIGWF

SEQ ID NO: 36 - protein, 17 aa, Synthetic construct
CISNIDGHTYYADSVKG

SEQ ID NO: 37 - protein, 15 aa, Synthetic construct
EGLHSCKDLYYGMDY

SEQ ID NO: 38 - protein, 124 aa, Synthetic construct
QVQLQESGGGLVQAGGSLRLSCAASGFTFDDYAIGWFRQAPGKEREGVSCISNIDGRTYYADSVKGRFTISSDNAKNTVYLQMNSLKPEDTAVYYCAAEGLHPCRDLYYGMDYWGKGTLVTVSS

SEQ ID NO: 39 - protein, 5 aa, Synthetic construct
AIGWF

SEQ ID NO: 40 - protein, 17 aa, Synthetic construct
CISNIDGRTYYADSVKG

SEQ ID NO: 41 - protein, 15 aa, Synthetic construct
EGLHPCRDLYYGMDY

SEQ ID NO: 42 - protein, 124 aa, Synthetic construct
QVQLQESGGGLVQAGGSLRLSCAASGFTFDDYAIGWFRQAPGKEREGVSCISNIDGRTYYADSVKGRFTISSDNAKNTVYLQMNSLKPEDTAVYYCAAEGLHSCRDLYYGMDYWGKGTLVTVSS

SEQ ID NO: 43 - protein, 5 aa, Synthetic construct
AIGWF

SEQ ID NO: 44 - protein, 17 aa, Synthetic construct
CISNIDGRTYYADSVKG

SEQ ID NO: 45 - protein, 15 aa, Synthetic construct
EGLHSCRDLYYGMDY

SEQ ID NO: 46 - protein, 124 aa, Synthetic construct
QVKLEESGGGLVQAGGSLRLSCAASGFTFDDYAIGWFRQAPGKEREGVSCISNIDGSTYYADSVKGRFTISSDNAKNTVYLQMNSLKPEDTAVYYCAAEGLHSCRDLYYGMDHWGKGTLVTVSS

SEQ ID NO: 47 - protein, 5 aa, Synthetic construct
AIGWF

SEQ ID NO: 48 - protein, 17 aa, Synthetic construct
CISNIDGSTYYADSVKG

SEQ ID NO: 49 - protein, 15 aa, Synthetic construct
EGLHSCRDLYYGMDH

SEQ ID NO: 50 - protein, 124 aa, Synthetic construct
QVQLVESGGGLVQAGGSLRLSCAASGFTFDDYAIGWFRQAPGKEREGVSCISNIDGSTYYADSVKGRFTISSDNAKNTVYLQMNSLKPEDTAVYYCAAEGLHSCRDLYYGMDYWGKGTLVTVSS

SEQ ID NO: 51 - protein, 5 aa, Synthetic construct
AIGWF

SEQ ID NO: 52 - protein, 17 aa, Synthetic construct
CISNIDGSTYYADSVKG

SEQ ID NO: 53 - protein, 15 aa, Synthetic construct
EGLHSCRDLYYGMDY

SEQ ID NO: 54 - protein, 124 aa, Synthetic construct
QVQLVESGGGLVQAGGSLRLSCAASGFTFDDYAIGWFRQAPGKEREGVSCISNIDGSTYYADSVKGRFTISSDNAKNTVYLQMNSLKPEDTAVYYCAAEGLRSCRDLYYGMDYWGKGTLVTVSS

SEQ ID NO: 55 - protein, 5 aa, Synthetic construct
AIGWF

SEQ ID NO: 56 - protein, 17 aa, Synthetic construct
CISNIDGSTYYADSVKG

SEQ ID NO: 57 - protein, 15 aa, Synthetic construct
EGLRSCRDLYYGMDY

SEQ ID NO: 58 - protein, 126 aa, Synthetic construct
QVQLVDSGGGLVQAGGSLRVSCAASGLTVSSEHMGWFRQAPGKQREFVAAISGNGGSTYYADSVKGRFTISRDNAKNTVYLQMNSLKPEDTAVYTCAADRQVWRVTSYHDYDYDSWGQGTLVTVSS

SEQ ID NO: 59 - protein, 5 aa, Synthetic construct
HMGWF

SEQ ID NO: 60 - protein, 17 aa, Synthetic construct
AISGNGGSTYYADSVKG

SEQ ID NO: 61 - protein, 17 aa, Synthetic construct
DRQVWRVTSYHDYDYDS

SEQ ID NO: 62 - protein, 126 aa, Synthetic construct
QVQLQESGGGLVQAGGSLRLSCAASGLTVNSDHMAWFRQAPGKWRDFVAAISGNGGSTYYADSVKGRFTISRDDAKNTVYLQMNSLKPEDTAVYTCAADRQVWRAASYHDYDYDSWGQGTLVTVSS

SEQ ID NO: 63 - protein, 5 aa, Synthetic construct
HMAWF

SEQ ID NO: 64 - protein, 17 aa, Synthetic construct
AISGNGGSTYYADSVKG

SEQ ID NO: 65 - protein, 17 aa, Synthetic construct
DRQVWRAASYHDYDYDS

SEQ ID NO: 66 - protein, 126 aa, Synthetic construct
QVKLEESGGGLVQAGDSLRLSCAASGLTVSSDHMAWFRQAPGKWREFVAAISGNGGSTYYADSVKGRFTISRDNAKNTVYLQMNNLKPEDTAVYSCAADRQVWRVASYHDYDYDSWGQGTQVTVSS

SEQ ID NO: 67 - protein, 5 aa, Synthetic construct
HMAWF

SEQ ID NO: 68 - protein, 17 aa, Synthetic construct
AISGNGGSTYYADSVKG

SEQ ID NO: 69 - protein, 17 aa, Synthetic construct
DRQVWRVASYHDYDYDS

SEQ ID NO: 70 - protein, 117 aa, Synthetic construct
QVQLQESGGGLVQAGGSLRLSCAASGSIAEIASLRAMDWYRQAPGKRRELVAHIARNDVTNYADDVKGRFTISRDNAKNAVYLQMNNLKPEDTAVYYCNTVPWYGTWGQGTLVTVSS

SEQ ID NO: 71 - protein, 5 aa, Synthetic construct
AMDWY

SEQ ID NO: 72 - protein, 16 aa, Synthetic construct
HIARNDVTNYADDVKG

SEQ ID NO: 73 - protein, 6 aa, Synthetic construct
VPWYGT

SEQ ID NO: 74 - protein, 117 aa, Synthetic construct
QVQLVESGGGLVQAGGSLRLSCAASGNIDEIASIRAMDWYRQAPGRQRELVAHIARNGITDYKDFVKGRFTISRDNAKNTVYLQMNSLKPEDTAVYYCNTVPWYGTWGQGTQVTVSS

SEQ ID NO: 75 - protein, 5 aa, Synthetic construct
AMDWY

SEQ ID NO: 76 - protein, 16 aa, Synthetic construct
HIARNGITDYKDFVKG

SEQ ID NO: 77 - protein, 6 aa, Synthetic construct
VPWYGT

SEQ ID NO: 78 - protein, 122 aa, Synthetic construct
EVQLVESGGGLVQPGGSLRLSCAASGFTFDEYAIGWFRQAPGKEREGVSCLRVSDGRTYYPDSVKGRFTISRDNSKNTVYLQMNSLRAEDTAVYYCASGSYYGCTVDDYDFWGQGTQVTVSS

SEQ ID NO: 79 - protein, 122 aa, Synthetic construct
EVQLQESGGGLVQAGGSLRLSCAASGFTFDEYAIGWVRQAPGKEREGVSCLRVSDGRTYYPDSVKGRFTISRDNSKNTVYLQMNNLKPEDTAVYYCASGSYYGCTVDDYDFWGQGTQVTVSS

SEQ ID NO: 80 - protein, 122 aa, Synthetic construct
EVQLQESGGGLVQPGGSLRLSCAASGFTFDEYAIGWVRQAPGKEREGVSCLRVSDGRTYYPDSVKGRFTISRDNSKNTVYLQMNSLRAEDTAVYYCASGSYYGCTVDDYDFWGQGTQVTVSS

SEQ ID NO: 81 - protein, 122 aa, Synthetic construct
EVQLQESGGGLVQPGGSLRLSCAASGFTFDEYAIGWVRQAPGKEREGVSCLRVSDGRTYYPDSVKGRFTISTDNSKNTVYLQMNSLRAEDTAVYYCASGSYYGCTVDDYDFWGQGTQVTVSS

SEQ ID NO: 82 - protein, 122 aa, Synthetic construct
QVQLQESGGGLVQAGGSLRLSCAASGFTFDEYAIGWVRQAPGKGLEGVSCLRVSDGRTYYPDSVKGRFAISSDNAKNTVYLQMNNLKPEDTAVYYCASGSYYGCTVDDYDFWGQGTQVTVSS

SEQ ID NO: 83 - protein, 122 aa, Synthetic construct
EVQLQESGGGLVQPGGSLRLSCAASGFTFDEYAIGWVRQAPGKGLEGVSCLRVSDGRTYYPDSVKGRFTISRDNAKNTVYLQMNNLKPEDTAVYYCASGSYYGCTVDDYDFWGQGTTVTVSS

SEQ ID NO: 84 - protein, 122 aa, Synthetic construct
EVQLQESGGGLVQAGGSLRLSCAASGFTFDEYAIGWVRQAPGKGLEGVSCLRVSDGRTYYPDSVKGRFTISRDNSKNTVYLQMNNLKPEDTAVYYCASGSYYGCTVDDYDFWGQGTQVTVSS

SEQ ID NO: 85 - protein, 122 aa, Synthetic construct
EVQLQESGGGLVQAGGSLRLSCAASGFTFDEYAIGWVRQAPGKGLEGVSCLRVSDGRTYYPDSVKGRFTISRDNSKNTVYLQMNNLKPEDTAVYYCASGSYYGCTVDDYDFWGQGTTVTVSS

SEQ ID NO: 86 - protein, 122 aa, Synthetic construct
QVQLQESGGGLVQAGGSLRLSCAASGFTFDEYAIGWFRQAPGKGLEGVSCLRVSDGRTYYPDSVKGRFAISSDNAKNTVYLQMNNLKPEDTAVYYCASGSYYGCTVDDYDFWGQGTQVTVSS

SEQ ID NO: 87 - protein, 122 aa, Synthetic construct
EVQLQESGGGLVQPGGSLRLSCAASGFTFDEYAIGWFRQAPGKGLEGVSCLRVSDGRTYYPDSVKGRFTISRDNAKNTVYLQMNNLKPEDTAVYYCASGSYYGCTVDDYDFWGQGTTVTVSS

SEQ ID NO: 88 - protein, 122 aa, Synthetic construct
EVQLQESGGGLVQAGGSLRLSCAASGFTFDEYAIGWFRQAPGKGLEGVSCLRVSDGRTYYPDSVKGRFTISRDNSKNTVYLQMNNLKPEDTAVYYCASGSYYGCTVDDYDFWGQGTQVTVSS

SEQ ID NO: 89 - protein, 122 aa, Synthetic construct
EVQLQESGGGLVQAGGSLRLSCAASGFTFDEYAIGWFRQAPGKGLEGVSCLRVSDGRTYYPDSVKGRFTISRDNSKNTVYLQMNNLKPEDTAVYYCASGSYYGCTVDDYDFWGQGTTVTVSS

SEQ ID NO: 90 - protein, 122 aa, Synthetic construct
QVQLQESGGGLVQAGGSLRLSCAASGFTFDEYAIGWVRQAPGKGREGVSCLRVSDGRTYYPDSVKGRFAISSDNAKNTVYLQMNNLKPEDTAVYYCASGSYYGCTVDDYDFWGQGTQVTVSS

SEQ ID NO: 91 - protein, 122 aa, Synthetic construct
EVQLQESGGGLVQPGGSLRLSCAASGFTFDEYAIGWVRQAPGKGREGVSCLRVSDGRTYYPDSVKGRFTISRDNAKNTVYLQMNNLKPEDTAVYYCASGSYYGCTVDDYDFWGQGTTVTVSS

SEQ ID NO: 92 - protein, 122 aa, Synthetic construct
EVQLQESGGGLVQAGGSLRLSCAASGFTFDEYAIGWVRQAPGKGREGVSCLRVSDGRTYYPDSVKGRFTISRDNSKNTVYLQMNNLKPEDTAVYYCASGSYYGCTVDDYDFWGQGTQVTVSS

SEQ ID NO: 93 - protein, 122 aa, Synthetic construct
EVQLQESGGGLVQAGGSLRLSCAASGFTFDEYAIGWVRQAPGKGREGVSCLRVSDGRTYYPDSVKGRFTISRDNSKNTVYLQMNNLKPEDTAVYYCASGSYYGCTVDDYDFWGQGTTVTVSS

SEQ ID NO: 94 - protein, 122 aa, Synthetic construct
QVQLQESGGGLVQAGGSLRLSCAASGFTFDEYAIGWFRQAPGKGREGVSCLRVSDGRTYYPDSVKGRFAISSDNAKNTVYLQMNNLKPEDTAVYYCASGSYYGCTVDDYDFWGQGTQVTVSS

SEQ ID NO: 95 - protein, 122 aa, Synthetic construct
EVQLQESGGGLVQPGGSLRLSCAASGFTFDEYAIGWFRQAPGKGREGVSCLRVSDGRTYYPDSVKGRFTISRDNAKNTVYLQMNNLKPEDTAVYYCASGSYYGCTVDDYDFWGQGTTVTVSS

SEQ ID NO: 96 - protein, 122 aa, Synthetic construct
EVQLQESGGGLVQAGGSLRLSCAASGFTFDEYAIGWFRQAPGKGREGVSCLRVSDGRTYYPDSVKGRFTISRDNSKNTVYLQMNNLKPEDTAVYYCASGSYYGCTVDDYDFWGQGTQVTVSS

SEQ ID NO: 97 - protein, 122 aa, Synthetic construct
EVQLQESGGGLVQAGGSLRLSCAASGFTFDEYAIGWFRQAPGKGREGVSCLRVSDGRTYYPDSVKGRFTISRDNSKNTVYLQMNNLKPEDTAVYYCASGSYYGCTVDDYDFWGQGTTVTVSS

SEQ ID NO: 98 - protein, 122 aa, Synthetic construct
QVQLQESGGGLVQAGGSLRLSCAASGFTFDEYAIGWVRQAPGKGLEWVSCLRVSDGRTYYPDSVKGRFAISSDNAKNTVYLQMNNLKPEDTAVYYCASGSYYGCTVDDYDFWGQGTQVTVSS

SEQ ID NO: 99 - protein, 122 aa, Synthetic construct
EVQLQESGGGLVQPGGSLRLSCAASGFTFDEYAIGWVRQAPGKGLEWVSCLRVSDGRTYYPDSVKGRFTISRDNAKNTVYLQMNNLKPEDTAVYYCASGSYYGCTVDDYDFWGQGTTVTVSS

SEQ ID NO: 100 - protein, 122 aa, Synthetic construct
EVQLQESGGGLVQAGGSLRLSCAASGFTFDEYAIGWVRQAPGKGLEWVSCLRVSDGRTYYPDSVKGRFTISRDNSKNTVYLQMNNLKPEDTAVYYCASGSYYGCTVDDYDFWGQGTQVTVSS

SEQ ID NO: 101 - protein, 122 aa, Synthetic construct
EVQLQESGGGLVQAGGSLRLSCAASGFTFDEYAIGWVRQAPGKGLEWVSCLRVSDGRTYYPDSVKGRFTISRDNSKNTVYLQMNNLKPEDTAVYYCASGSYYGCTVDDYDFWGQGTTVTVSS

SEQ ID NO: 102 - protein, 122 aa, Synthetic construct
QVQLQESGGGLVQAGGSLRLSCAASGFTFDEYAIGWFRQAPGKGLEWVSCLRVSDGRTYYPDSVKGRFAISSDNAKNTVYLQMNNLKPEDTAVYYCASGSYYGCTVDDYDFWGQGTQVTVSS

SEQ ID NO: 103 - protein, 122 aa, Synthetic construct
EVQLQESGGGLVQPGGSLRLSCAASGFTFDEYAIGWFRQAPGKGLEWVSCLRVSDGRTYYPDSVKGRFTISRDNAKNTVYLQMNNLKPEDTAVYYCASGSYYGCTVDDYDFWGQGTTVTVSS

SEQ ID NO: 104 - protein, 122 aa, Synthetic construct
EVQLQESGGGLVQAGGSLRLSCAASGFTFDEYAIGWFRQAPGKGLEWVSCLRVSDGRTYYPDSVKGRFTISRDNSKNTVYLQMNNLKPEDTAVYYCASGSYYGCTVDDYDFWGQGTQVTVSS

SEQ ID NO: 105 - protein, 122 aa, Synthetic construct
EVQLQESGGGLVQAGGSLRLSCAASGFTFDEYAIGWFRQAPGKGLEWVSCLRVSDGRTYYPDSVKGRFTISRDNSKNTVYLQMNNLKPEDTAVYYCASGSYYGCTVDDYDFWGQGTTVTVSS

SEQ ID NO: 106 - protein, 243 aa, Synthetic construct
EPKTPKPQPQPQPQPNPTTESKCPKCPAPELLGGPSVFIFPPKPKDVLSISGRPEVTCVVVDVGQEDPEVSFNWYIDGAEVRTANTRPKEEQFNSTYRVVSVLPIQHQDWLTGKEFKCKVNNKALPAPIEKTISKAKGQTREPQVYALAPHREELAKDTVSVTCLVKGFYPPDINVEWQRNRQPEPEGTYATTPPQLDNDGTYFLYSKLSVGKNTWQRGETFTCVVMHETLHNHYTQKSISQS

SEQ ID NO: 107 - protein, 227 aa, Synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 161 aa, Macaca fascicularis
NQFRVSPLGRTWNLGETVELKCQVLLSNPTSGCSWLFQPRGTAARPTFLLYLSQNKPKAAEGLDTQRFSGKRLGDTFVLTLRDFRQENEGYYFCSALSNSIMYFSHFVPVFPCSEAHHYASAATTHTGAHHRVAAPVPAPRGVPASGGGSVNTRGLDFACD